(19) United States
(12) Patent Application Publication
(10) Pub. No.: US 2002/0076747 A1
(43) Pub. Date: Jun. 20, 2002

(54) Method for expanding embryonic stem cells in serum-free culture

(76) Inventors: PAUL J. PRICE, GRAND ISLAND, NY (US); MINDY D. GOLDSBOROUGH, GAITHERSBURG, MD (US); MARY LYNN TILKINS, NIAGARA FALLS, NY (US)

Correspondence Address: STERNE KESSLER GOLDSTEIN & FOX, 1100 NEW YORK AVENUE N W, SUITE 600, WASHINGTON, DC, 200053934

(21) Appl. No.: 09/390,634

(22) Filed: Sep. 7, 1999

Related U.S. Application Data
(62) Division of application No. 08/781,772, filed on Jan. 10, 1997, now abandoned.

(51) Int. Cl. C12P 21/02; C07H 21/04; C12N 5/02; C12N 5/06

(52) U.S. Cl. 435/69.1; 435/325; 435/374; 435/375; 435/377; 435/395; 435/404; 435/405; 435/406; 435/407; 435/455; 536/23.1

(57) ABSTRACT

The present invention provides a serum-free supplement which supports the growth of embryonic stem cells in culture. Also provided are a medium comprising a basal medium supplemented with the serum-free supplement of the present invention. The present invention also provides methods for culturing and isolating embryonic stems cells, methods for producing a transgenic animal, and methods for expressing recombinant protein in embryonic stem cells and transgenic animals.

### FIELD OF THE INVENTION

The present invention relates to a replacement for the serum supplementation normally required for the isolation and proliferation of embryonic stem (ES) cells and other cell types, such as hybridomas.

### BACKGROUND OF THE INVENTION

ES cells are established cell lines derived from the inner cell mass of a blastocyst. The undifferentiated cells are pluripotent and take part in the formation of all tissues, including the germ line. After injection into blastocysts or morulae, or after aggregation with morulae (Wood, S. A., et al., Proc. Natl. Acad. Sci. USA 90:4582-4585 (1993)), ES cells generate offspring containing two different genomes (i.e., chimeric offspring). Breeding of chimeric animals having ES populated germ cells can result in the establishment of a line that is homozygous for the ES cell genome.

Using homologous recombinant technology and ES cells, researchers can introduce, in a targeted fashion, site-specific mutations into the genome. This technology facilitates the study of gene function and regulation in the resulting transgenic animal (Capecchi, M. R., Science 244:1288-1292 (1989)). In addition to gene targeting studies, ES cells have many applications for medical research, including the production of animal models of human disease (Smithies, O. et al., Proc. Natl. Acad. Sci. USA:5266-5272 (1995)) and as a model to study the process of cell differentiation (Doetschman, T. C. et al., J. Embryol. Exp. Morph. 87:27-45 (1985)).

ES cells are usually passaged onto a pre-plated layer of inactivated feeder cells, either primary embryonic fibroblasts or STO cells. Feeder cells provide a matrix for ES cell attachment. Moreover, by contributing undefined growth factors, feeder cells play an important role in preventing ES cells from differentiating in culture.

When using ES cells for gene targeting or for use as cell precursors, it is imperative to preserve the embryonic, pleuripotential (i.e., non-differentiated) phenotype of the ES cells. In addition to feeder cells, many researchers also use leukemia inhibitory factor (LIF), or other growth factors, to prevent cultured ES cells from differentiating (Smith, A. G., Nature 336:688-690 (1988); Gearing, D.P. et al., U.S. Pat. No. 5,187,077 (1993)). Researchers presently use feeder cell layers, in combination with LIF, in order to maintain the pluripotency of ES cells in vitro. However, some ES lines have been developed that do not require feeder cell layers. Instead, these feeder-cell independent ES cells are seeded onto gelatinized petri plates (Magin, T. M., Nucl. Acids, Res. 20:3795-3796 (1992)). Generally, feeder-cell independent ES cell lines are cultured in medium supplemented with growth factors (e.g., LIF). Moreover, to assist in avoiding ES cell differentiation, ES cells generally are not maintained in culture for periods of time longer than absolutely necessary.

To aid in evaluating culture conditions, assay methods utilizing cell differentiation markers have been developed. Several cell markers, including alkaline phosphatase, can be used to distinguish undifferentiated cells from those that have undergone differentiation (Pease, S. et al., Devel. Biol. 141:344-352 (1990)).

Yet, because ES cells are typically cultured in medium supplemented with serum (e.g., fetal bovine serum (FBS)), ES cells tend to differentiate. Serum is a major source of undefined differentiation factors and thus tends to promote ES cell differentiation. Other problems are also associated with serum. Lot-to-lot variation is often observed and some lots of serum have been found to be toxic to cells (Robertson, E. J., ed., Teratocarcinomas and Embryonic Stem Cells: A Practical Approach, IRL Press, Oxford, UK (1987)). Moreover, serum may be contaminated with infectious agents such as mycoplasma, bacteriophage, and viruses. Finally, because serum is an undefined and variable component of any medium, the use of serum prevents the true definition and elucidation of the nutritional and hormonal requirements of the cultured cells.

In view of the many problems associated with the use of serum in the growth of ES cells, laboratories performing work with ES cells must resort to pre-screening serum prior to purchase. However, the pre-screening process is time-consuming and subject to interpretation. Even after a satisfactory lot is identified, storage of large quantities of pre-screened lots of serum at −20° C. and below is problematic.

Thus, research with ES cells, such as the isolation of ES cells, cultivation of ES cells in culture, expansion of ES cells, control of differentiation of ES cells, and explantation of ES cells, is hindered by the necessity for serum. Thus, there remains a need for a serum-free medium supplement and a serum-free medium which supports the growth and expansion of ES cells without promoting or inducing the differentiation of ES cells in culture.

### SUMMARY OF THE INVENTION

The present invention provides a serum-free, eukaryotic cell culture medium supplement, wherein a basal cell culture medium supplemented with the serum-free supplement is capable of supporting the growth of ES cells in serum-free culture.

The serum-free eukaryotic cell culture medium supplement comprises or is obtained by combining one or more ingredients selected from the group consisting of albumins or albumin substitutes, one or more amino acids, one or more vitamins, one or more transferrins or transferrin substitutes, one or more antioxidants, one or more insulins or insulin substitutes, one or more collagen precursors, and one or more trace elements. Preferably, the supplement of the present invention comprises an albumin or an albumin substitute and one or more ingredients selected from group consisting of one or more amino acids, one or more vitamins, one or more transferrins or transferrin substitutes, one or more antioxidants, one or more insulins or insulin substitutes, one or more collagen precursors, and one or more trace elements.

The present invention specifically provides a serum-free, eukaryotic cell culture medium supplement comprising or obtained by combining Albumax® I and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr3+, Ge⁴⁺, Se⁴⁺, Br⁻, I⁻, Mn²⁺, F⁻, Si⁴⁺, V⁵⁺, Mo⁶⁺, Ni²⁺, Rb⁺, Sn²⁺ and Zr⁴⁺.

The present invention also provides a eukaryotic cell culture medium comprising a basal cell culture medium supplemented with the serum-free cell culture supplement of the invention. The present invention also provides a eukaryotic cell culture medium obtained by combining a basal cell culture medium with the serum-free supplement of the invention.

The present invention also provides a method of making a serum-free eukaryotic cell culture medium, the method comprising mixing the supplement of the invention and a basal medium. The present invention also provides a method of making the serum-free eukaryotic cell culture medium supplement.

The present invention also provides a composition comprising ES cells and the supplement of the invention. The present invention also provides a composition comprising ES cells and a serum-free medium, wherein the serum-free medium is capable of supporting the growth of ES cells in serum-free culture.

The present invention also provides a product of manufacture comprising a container means containing ES cells and the supplement of the invention. The present invention also provides a product of manufacture comprising a container means containing ES cells and the serum-free medium of the invention. The present invention also provides a product of manufacture comprising one or more container means, wherein a first container means contains the supplement of the invention or a serum-free medium of the invention. Optionally, a second container means contains a basal medium. Optionally, a third container means contains ES cells.

The present invention also provides a method of expanding ES cells in serum-free culture, the method comprising contacting ES cells with a serum-free medium capable of supporting the growth of ES cells in serum-free culture, and cultivating the ES cells under serum-free conditions suitable to facilitate the expansion of the ES cells. The present invention also provides a population of expanded ES cells obtained by this method.

The present invention also provides a method of producing a transgenic animal, the method comprising cultivating ES cells in serum-free culture, introducing a nucleic acid molecule into ES cells, selecting a recombinant ES cell clone, expanding the recombinant ES cell clone to form a population, injecting an aliquot of the recombinant ES cell clonal population into a blastocyst, transferring the injected blastocyst into a host pseudopregnant female animal, and selecting transgenic offspring. The present invention also provides a transgenic animal obtained by this method.

The present invention also provides a method of producing a transgenic animal, the method comprising cultivating ES cells in serum-free culture, introducing a nucleic acid molecule into ES cells, selecting a recombinant ES cell clone, expanding the recombinant ES cell clone to form a population, co-culturing a small number of the ES cells with early stage embryos (e.g., eight cell morulae) to form aggregates of embryos, transferring the aggregated embryos into a host pseudopregnant female animal, and selecting transgenic offspring. The present invention also provides a transgenic animal obtained by this method.

The present invention also provides a method of producing a recombinant protein from a transgenic animal, the method comprising cultivating ES cells in serum-free culture, introducing a nucleic acid construct comprising a nucleic acid molecule which encodes a protein of interest into the ES cells, selecting a recombinant ES cell clone, expanding the recombinant ES cell clone to form a population, injecting the recombinant ES cell clonal population into a blastocyst, transferring the injected blastocyst into a host pseudopregnant female animal, selecting a transgenic offspring, raising the selected transgenic animal(s) under conditions suitable to promote the health of the animal, and isolating the recombinant protein from the transgenic animal. The present invention also provides a protein obtained by this method.

The present invention also provides a method of producing a recombinant protein from a transgenic animal, the method comprising cultivating ES cells in serum-free culture, introducing a nucleic acid construct comprising a nucleic acid molecule which encodes a protein of interest into ES cells, selecting a recombinant ES cell clone, expanding the recombinant ES cell clone to form a population, co-culturing a small number of the ES cells with early stage embryos (e.g., eight cell morulae) to form aggregates of embryos, transferring the aggregated embryos into a host pseudopregnant female animal, selecting transgenic offspring, raising the selected transgenic animal(s) under conditions suitable to promote the health of the animal, and isolating the recombinant protein from the transgenic animal. The present invention also provides a recombinant protein obtained by this method.

The present invention also provides a method for controlling or preventing the differentiation of ES cells in serum-free culture. The method comprises contacting ES cells with the serum-free culture medium of the present invention, and cultivating the ES cells under serum-free conditions suitable to prevent the differentiation of the ES cells and facilitate the expansion of ES cells in serum-free culture.

The present invention also provides a method of causing ES cells to differentiate into a particular type of cell in serum-free culture. The method comprises contacting ES cells with a serum-free culture medium, culturing the ES cells under serum-free conditions suitable to facilitate the expansion of ES cells in serum-free culture, and adding a differentiation factor or changing culturing conditions to induce differentiation of ES cells to form a different type or a particular type of cell.

The present invention also provides a method of providing differentiated ES cells to a mammal. The method comprises contacting ES cells with a serum-free culture medium, culturing the ES cells under serum-free conditions suitable to facilitate the expansion of ES cells in serum-free culture, adding a differentiation factor or changing culturing conditions to induce differentiation of ES cells to form a different type or a particular type of cell, and introducing the differentiated ES cells into a mammal.

The present invention also provides a method of obtaining ES cells in serum-free culture. The method comprises isolating ES cells from cultured blastocysts, and cultivating the isolated ES cells in serum-free culture under conditions suitable to facilitate ES cell expansion and prevent ES cell differentiation. The present invention also provides ES cells obtained by the method.

The present invention also provides a method of producing recombinant protein in serum-free culture. The method comprises obtaining a recombinant eukaryotic cell (e.g., an ES cell or hybridoma) containing a nucleic acid construct comprising a nucleic acid molecule which encodes a protein of interest, culturing the cell in serum free culture to form a population of cells, and isolating the protein from said cells or from the medium in which the cells are cultured. The present invention also provides a recombinant protein obtained by the method.

### BRIEF DESCRIPTION OF THE FIGURES

All photographs were taken on a Nikon Diaphot-TMD phase contrast microscope at 100× magnification.

FIG. 1A shows ES cell colonies after 7 days of growth in DMEM supplemented with L-glutamine, non-essential amino acids (NEAA), 2-mercaptoethanol, penicillin/streptomycin, LIF (10 ng/mL) and 15% FBS.

FIG. 1B shows ES colonies after fixation and staining for the detection of alkaline phosphatase activity. Culture conditions were the same as in FIG. 1A.

FIG. 2A shows ES cell colonies after 7 days of growth in DMEM supplemented with L-glutamine, NEAA, 2-mercaptoethanol, penicillin/streptomycin, LIF (10 ng/mL) and a 15% concentration of the serum-free supplement of the present invention.

FIG. 2B shows ES cell colonies after fixation and staining for the detection of alkaline phosphatase activity. Culture conditions were the same as in FIG. 2A.

### DETAILED DESCRIPTION OF THE INVENTION

In the description that follows, a number of terms conventionally used in the field of cell culture media and for the growth of eukaryotic cells are utilized extensively. In order to provide a clear and consistent understanding of the specification and claims, and the scope to be given such terms, the following definitions are provided.

The term “albumin substitute” refers to any compound which may be used in place of albumin (e.g., bovine serum albumin (BSA) or AlbuMAX® I) in the supplement of the invention to give substantially similar results as albumin. Albumin substitutes may be any protein or polypeptide source. Examples of such protein or polypeptide samples include but are not limited to bovine pituitary extract, plant hydrolysate (e.g., rice hydrolysate), fetal calf albumin (fetuin), egg albumin, human serum albumin (HSA), or another animal-derived albumins, chick extract, bovine embryo extract, AlbuMAX® I, and AlbuMAX® II. Preferably, the albumin substitute is AlbuMAX® I. In the supplement and the medium of the present invention, the concentration of albumin or albumin substitute which facilitates cell culture can be determined using only routine experimentation.

The term “transferrin substitute” refers to any compound which may replace transferrin in the supplement of the invention to give substantially similar results as transferrin. Examples of transferrin substitutes include but are not limited to any iron chelate compound. Iron chelate compounds which may be used include but are not limited to iron chelates of ethylenediaminetetraacetic acid (EDTA), ethylene glycol-bis(β-aminoethyl ether)-N,N,N′,N′-tetraacetic acid (EGTA), deferoxamine mesylate, dimercaptopropanol, diethylenetriamine-pentaacetic acid (DPTA), and trans-1,2-diaminocyclohexane-N,N,N′,N′-tetraacetic adic (CDTA), as well as a ferric citrate chelate and a ferrous sulfate chelate. Preferably, the transferrin substitute is a ferric citrate chelate or a ferrous sulfate chelate. Most preferably, the transferrin substitute is the iron chelate ferrous sulphate.7 water.EDTA. In the supplement and the medium of the present invention, the concentration of the transferrin substitute which facilitates cell culture can be determined using only routine experimentation.

The term “insulin substitute” refers to any zinc containing compound which may be used in place of insulin in the supplement of the invention to give substantially similar results as insulin. Examples of insulin substitutes include but are not limited to zinc chloride, zinc nitrate, zinc bromide, and zinc sulfate. Preferably, the insulin substitute is zinc sulfate.7 water. In the supplement and the medium of the present invention, the concentration of the insulin substitute which facilitates cell culture can be determined using only routine experimentation.

The term “expand” refers to the growth and division, and not the differentiation of ES cells in culture.

The term “collagen precursor” refers to any compound which is utilized by cells to synthesize collagen. Collagen precursors which may be used in the supplement or the medium of the present invention include but are not limited to L-proline, L-hydroxyproline, and multimers or derivatives thereof, and ascorbic acid and derivatives thereof. One or more of such compounds may be used for the formation of collagen.

The term “antioxidant” refers to molecules which inhibit reactions that are promoted by oxygen or peroxides. Antioxidants which may be used in the supplement or the medium of the present invention include but are not limited to reduced glutathione and ascorbic acid-2-phosphate or derivatives thereof.

The term “ingredient” refers to any compound, whether of chemical or biological origin, that can be used in cell culture media to maintain or promote the growth or proliferation of cells. The terms “component,” “nutrient” and “ingredient” can be used interchangeably and are all meant to refer to such compounds. Typical ingredients that are used in cell culture media include amino acids, salts, metals, sugars, lipids, nucleic acids, hormones, vitamins, fatty acids, proteins and the like. Other ingredients that promote or maintain growth of cells er vivo can be selected by those of skill in the art, in accordance with the particular need.

By “cell culture” is meant cells or tissues that are maintained, cultured or grown in an artificial, in vitro environment.

By “culture vessel” it is meant glass containers, plastic containers, or other containers of various sizes that can provide an aseptic environment for growing cells. For example, flasks, single or multiwell plates, single or multiwell dishes, or multiwell microplates can be used.

The terms “cell culture medium,” “culture medium” and “medium formulation” refer to a nutritive solution for culturing or growing cells.

The terms “cultivating” and “culturing” are synonymous.

The term “container means” includes culture vessels, jars, bottles, vials, straws, ampules, and cryotubes.

The term “feeding” or “fluid-changing” refers to replacing the medium in which cells are cultured.

The term “combining” refers to the mixing or admixing of ingredients in a cell culture medium formulation.

The term “contacting” refers to the mixing, adding, seeding, or stirring of one or more cells with one or more compounds, solutions, media, etc.

A “serum-free” medium is a medium that contains no serum (e.g., fetal bovine serum (FBS), horse serum, goat serum, etc.).

By “compatible ingredients” is meant those media nutrients which can be maintained in solution and form a “stable” combination. A solution containing “compatible ingredients” is said to be “stable” when the ingredients do not degrade or decompose substantially into toxic compounds, or do not degrade or decompose substantially into compounds that cannot be utilized or catabolized by the cell culture. Ingredients are also considered “stable” if degradation can not be detected or when degradation occurs at a slower rate when compared to decomposition of the same ingredient in a 1× cell culture media formulation. Glutamine, for example, in 1× X media formulations, is known to degrade into pyrolidone carboxylic acid and ammonia. Glutamine in combination with divalent cations are considered “compatible ingredients” since little or no decomposition can be detected over time. See U.S. Pat. No. 5,474,931.

A cell culture medium is composed of a number of ingredients and these ingredients vary from medium to medium. Each ingredient used in a cell culture medium has unique physical and chemical characteristics. Compatibility and stability of ingredients are determined by the “solubility” of the ingredients in solution. The terms “solubility” and “soluble” refer to the ability of an ingredient to form a solution with other ingredients. Ingredients are thus compatible if they can be maintained in solution without forming a measurable or detectable precipitate. Thus, the term “compatible ingredients” as used herein refers to the combination of particular culture media ingredients which, when mixed in solution either as concentrated or IX formulations, are “stable” and “soluble.”

A “1× formulation” is meant to refer to any aqueous solution that contains some or all ingredients found in a cell culture medium. The “1× formulation” can refer to, for example, the cell culture medium of any subgroup of ingredients for that medium. The concentration of an ingredient in a 1× solution is about the same as the concentration of that ingredient found in the cell culture formulation used for maintaining or growing cells. Briefly, a culture medium used to grow cells is, by definition, a 1× formulation. When a number of ingredients are present (as in a subgroup of compatible ingredients), each ingredient in a 1× formulation has a concentration about equal to the concentration of those ingredients in a cell culture medium. For example, RPMI 1640 culture medium contains, among other ingredients, 0.2 g/L L-arginine, 0.05 g/L L-asparagine, and 0.02 g/L L aspartic acid. A “1× formulation” of these amino acids, which are compatible ingredients according to the present invention, contains about the same concentrations of these ingredients in solution. Thus, when referring to a “1× formulation,” it is intended that each ingredient in solution has the same or about the same concentration as that found in the cell culture medium being described. The concentrations of medium ingredients in a 1× formulation are well known to those of ordinary skill in the art. See Methods For Preparation of Media, Supplements and Substrate For Serum-Free Animal Cell Culture, Allen R. Liss, N.Y. (1984), which is incorporated by reference herein in its entirety.

A 10× formulation refers to a solution wherein each ingredient in that solution is about 10 times more concentrated than the same ingredient in the cell culture media. RPMI 1640 media, for example, contains, among other things, 0.3 g/L L-glutamine. A “10× formulation” may contain a number of additional ingredients at a concentration about 10 times that found in the 1× culture media. As will be apparent, “25X formulation,” “50× formulation,” and “100× formulation” designate solutions that contain ingredients at about 25, 50 or 100 fold concentrations, respectively, as compared to a 1× cell culture media.

The term “trace element” or “trace element moiety” refers to a moiety which is present in a cell culture medium in only trace amounts. In the present invention, these terms encompass Ag⁺, Al³⁺, Ba²⁺, Cd²⁺, Co²⁺, Cr3+, Ge⁴⁺, Se⁴⁺, Br⁻, I⁻, Mn²⁺, F⁻, Si⁴⁺, V⁵⁺, Mo⁶⁺, Ni²⁺, Rb⁺, Sn²⁺ and Zr⁴⁺ and salts thereof. Suitable concentrations of trace element moieties can be determined by one of ordinary skill in the art (See Table 2).

Any salt of a given trace element moiety can be used to make the supplement or the medium of the present invention. For example, the following salts can be used: AgNO₃, AlCl₃.6H₂O, Ba(C₂H₃O₂)₂, CdSO₄.8H₂O, CoCl₂.6 H₂O, Cr₂(SO₄)₃.1H₂O, GeO₂, Na₂SeO₃, H₂SeO₃, KBr, KI, MnCl₂.4H₂O, NaF, Na₂SiO₃.9H₂O, NaVO₃, (NH₄)₆Mo₇O₂₄.4H₂O, NiSO₄.6H₂O, RbCl, SnCl₂, and ZrOCl₂.8H₂O. Suitable concentrations of trace element moiety-containing compounds can be determined by one of ordinary skill in the art (See Table 3).

Examples of concentrations of compounds containing selenium, silicon, vanadium, molybdenum, and zirconium are as follows. In a preferred embodiment of the supplement of the invention, the concentration of SeO₃²⁻ is about 0.02 mg/L, the concentration of SiO₃²⁻ is about 0.3 mg/L, the concentration of VO₃⁻ is about 0.005 mg/L, the concentration of Mo₇O₂₄⁶⁻ is about 0.05 mg/L, and the concentration of ZrO²⁺ is about 0.005 mg/L. In the 1× medium of the present invention, the concentration rage of SeO₃²⁻ is about 0.00001 to about 0.007 mg/L, the concentration range of SiO₃²⁻ is about 0.0003 to about 0.3 mg/L, the concentration range Of VO₃⁻ is about 0.000008 to about 0.008 mg/L, the concentration range of Mo₇O₂₄⁶⁻ is about 0.000009 to about 0.09 mg/L, and the concentration range of ZrO²⁺ is about 0.00006 to about 0.006 mg/L. In a preferred embodiment of the 1× medium, the concentration of SeO₃²⁻ is about 0.003 mg/L, the concentration of SiO₃²⁻ is about 0.04 mg/L, the concentration of VO₃⁻ is about 0.0007 mg/L, the concentration of Mo₇O₂₄⁶⁻ is about 0.008 mg/L, and the concentration of ZrO²⁺ is about 0.0008 mg/L.

The term “amino acid” refers to amino acids or their derivatives (e.g., amino acid analogs), as well as their D- and L-forms. Examples of such amino acids include glycine, L-alanine, L-asparagine, L-cysteine, L-aspartic acid, L-glutamic acid, L-phenylalanine, L-histidine, L-isoleucine, L-lysine, L-leucine, L-glutamine, L-arginine, L-methionine, L-proline, L-hydroxyproline, L-serine, L-threonine, L-tryptophan, L-tyrosine, and L-valine.

The terms “embryonic stem cell” and “pluripotent embryonic stem cell” refer to a cell which can give rise to many differentiated cell types in an embryo or an adult, including the germ cells (sperm and eggs). This cell type is also referred to as an “ES” cell herein.

A “population” of ES cells refers to any number of ES cells greater than one. Similarly, a population of blastocysts refers to any number of blastocysts greater than one.

The terms “recombinant embryonic stem cell” or a “recombinant embryonic stem cell clone” refer to an ES cell into which a nucleic acid molecule has been introduced and has become stably maintained. The nucleic acid molecule can contain a drug resistance gene which aids in the selection of recombinant ES cells. After introduction of the nucleic acid molecule and clonal drug selection, ES clones are analyzed by either PCR or Southern blotting methods to verify correct gene targeting.

The term “nucleic acid construct” refers to a nucleic acid molecule which contains a nucleic acid that encodes a protein of interest. Preferably, the nucleic acid construct is an expression vector which contains the nucleic acid encoding the protein of interest operably linked to an expression control sequence (i.e., a promoter and/or an enhancer, regulatory sequences to which gene regulatory proteins bind and exert control over gene transcription). Expression vectors which may be used are well known to those of ordinary skill in the art.

The term “basal medium” refers to any medium which is capable of supporting growth of ES cells, or other cells, when supplemented either with serum or with the serum-free supplement of the present invention. The basal medium supplies standard inorganic salts, such as zinc, iron, magnesium, calcium and potassium, as well as vitamins, glucose, a buffer system, and essential amino acids. Basal media which can be used in the present invention incude but are not limited to Dulbecco's Modified Eagle's Medium (DMEM), Minimal Essential Medium (MEM), Basal Medium Eagle (BME), RPMI 1640, F-10, F-12, α Minimal Essential Medium (αMEM), Glasgow's Minimal Essential Medium (G-MEM), and Iscove's Modified Dulbecco's Medium. In a preferred embodiment, the basal medium is DMEM with high glucose, either with or without the sodium salt of pyruvic acid. Pyridoxine.HCl can be used in place of pyridoxal.

The terms “serum-free culture conditions” and “serum-free conditions” refer to cell culture conditions that exclude serum of any type.

The present invention provides a substitute for the serum component of a complete medium for the establishment and growth of ES cells and other cell types. The serum-free eukaryotic cell culture medium supplement comprises or is obtained by combining one or more ingredients selected from the group consisting of albumins or albumin substitutes, one or more amino acids, one or more vitamins, one or more transferrins or transferrin substitutes, one or more antioxidants, one or more insulins or insulin substitutes, one or more collagen precursors, and one or more trace elements. Preferably, the supplement of the present invention comprises an albumin or an albumin substitute and one or more ingredients selected from group consisting of one or more amino acids, one or more vitamins, one or more transferrins or transferrin substitutes, one or more antioxidants, one or more insulins or insulin substitutes, one or more collagen precursors, and one or more trace elements.

Specifically, the supplement of the present invention is comprised of a lipid-rich bovine serum albumin or albumin substitute (Albumax® I, available from Life Technologies, Gaithersburg, Md.), and one or more ingredients selected from the group consisting of one or more amino acids, one or more vitamins, one or more of transferrin or a transferrin substitute, one or more antioxidants (e.g., glutathione and L-ascorbic acid-2-phosphate), one or more of insulin or an insulin substitute, one or more collagen precursors, and one or more trace elements. L-ascorbic acid-2-phosphate, in combination with L-proline and L-hydroxyproline, is also important as a collagen precursor. The supplement of the present invention can be added to any basal medium. When added to a basal medium, such as Dulbecco's modified Eagle's medium (DMEM) with high glucose (available from Life Technologies, Gaithersburg, Md.), the supplement of the present invention supports the growth of undifferentiated ES cells and hybridoma cells to an extent equal to, or better than, fetal bovine serum (FBS) qualified for either ES cell or hybridoma growth.

In most laboratories, the standard medium combination used to grow and passage ES cell cultures is DMEM (high glucose) supplemented with 15% pretested and heat-inactivated FBS, 100 μM 2-mercaptoethanol, and 100 μM non-essential amino acids (NEAA). For the establishment of ES cell cultures, nucleosides are sometimes added to the medium (Robertson, E. J., ed., Teratocarcinomas and Embryonic Stem Cells: A Practical Approach, IRL Press, Oxford, UK (1987)). The supplement of the present invention is added to the basal medium, in place of the serum (e.g., FBS) component, and at the same final percentage as serum, usually about 15% in ES cell cultures. However, the final concentration of the supplement of the present invention can be from about 0.5% to about 90%. Preferably, the final concentration of the supplement is from about 5% to about 50%. More preferably, the final concentration of the supplement is from about 5% to about 30%. Still more preferably, the final concentration of the supplement is about 5% to about 20%. The most preferred final concentration of the supplement is about 15%.

Due to its defined and reproducible composition, the supplement of the present invention does not require pretesting for suitability. Moreover, since no complement factors are present in the supplement of the present invention, it does not require heat-inactivation.

ES cells find major use in the production of transgenic animals containing site-specific modifications in their genomes. In order to alter the genetic makeup of the ES cells, a nuleic acid molecule or construct containing a genetically altered copy of the gene is introduced into ES cells. The introduction of nucleic acid into ES cells has been achieved in many ways, including precipitation with calcium phosphate (Gossler, A. et al., Proc. Natl. Acad. Sci. USA:9065-9069 (1989)), retrovirus infection (Robertson, E., et al., Nature 323:445-448 (1986)), electroporation (Thompson, S. et al., Cell 56:313-321 (1989)) and cationic lipids (Lamb, B. T., et al., Nature Genetics 5:22-29 (1993)).

In a fraction of the ES cells which take up the nucleic acid molecule or construct, the introduced nucleic acid molecule or construct undergoes homologous recombination with the native copy of that gene. A suitable selection gene (or genes) is incorporated into the nucleic acid molecule or construct to allow drug selection of recombinant ES cells via the addition of the selection drug(s) into the culture medium. After introduction of the nucleic acid molecule or construct and clonal drug selection, ES clones are analyzed by either PCR or Southern blotting methods to verify correct gene targeting.

Next, selected ES clones are injected into blastocysts. The goal is for the 15 or so injected recombinant ES cells to mix with the resident inner cell mass of the blastocyst and result in a chimeric offspring. Injected blastocysts are transferred into host pseudopregnant females for gestation.

The progress of the experiment can be monitored at birth through the use of markers. For example, in mice, almost all ES cell lines are presently derived from the 129 strain of mice (having an agouti coat color). The host blastocysts are generally derived from C57B1/6 mice (having a black coat color). A chimeric animal with a good proportion of ES cell-derived tissues will generally be male (ES cell lines are male) and have predominantly agouti coat color.

The predominance of male offspring is the result of sex conversion of female embryos by the male ES cell lines (Robertson, E. J. et al., J. Embryol. Exp. Morph. 74:297-309 (1983)). However, female chimeras that transmit to the germline are also sometimes produced (Lamb, B. T., et al., Nature Genetics 5:22-29 (1993)). In order to test whether the chimeric animals have the targeted gene in their germline, they are backcrossed to C57B 1/6 mates (where the agouti coat color is dominant over the black coat color). If agouti pups are produced, then a germline transmission of the ES derived genome will have occurred. Such offspring will be heterozygous for the ES genome. If desired, heterozygous animals can be interbred to establish a homozygous population of targeted animals.

The gene targeting process requires that a germline competent ES cell line be used. This line may be obtained from scientific collaborators, from a commercial source (e.g., American Type Culture Collection, Rockville, Md.; Genome Systems, Inc., St. Louis, Mo.; Lexicon Genetics, Inc., Woodlands, Tex.), or can be developed by the individual investigator. The present invention may be used for the isolation of ES lines in the following manner. ES cell lines are established from blastocyst staged embryos by allowing the inner cell mass to grow out from embryos placed on top of a feeder layer of inactivated mouse embryo fibroblasts or STO cells. Multiple blastocyts are initiated at any particular time, as only a small percent of the initiated cultures will form germline competent ES cell lines.

Unwanted cell differentiation, absence of an XY karyotype, and poor ES cell and colony morphology are among the main reasons why the majority of the potential ES cultures do not serve as effective ES cell lines. As with general ES cell culture, the undefined factors present in serum (e.g., FBS) can have a dramatic negative effect on the establishment of ES cell lines. Accordingly, the supplement or the medium of the present invention can be used as a substitute for serum for ES cell line establishment. Due to its defined composition and lack of uncharacterized differentiation factors, the supplement and the medium of the present invention increase the likelihood of establishing an ES cell line.

Moreover, the supplement or the medium of the present invention is important in the establishment of true, germline competent, ES cells from murine and non-murine species. In establishing such ES cell lines, the supplement or the medium of the present invention is used alone or in conjunction with general or species specific growth factors.

According to the invention, an ES cell line can be obtained from any animal. Examples of animals from which blastocysts and ES cells can be isolated using the supplement and the medium of the present invention include mouse (Evans, M. J. et al., Nature 292:154-156 (1981)), rat (Iannaccone, P. M. et al., Devel. Biol. 163:288-292 (1994)), hamster (Doetschman, T. et al., Devel. Biol. 127:224-227 (1988)), rabbit (Graves, K. H. et al., Molec. Reprod. Devel. 36:424-433 (1993)), monkey (Thomson, J. A. et al., Proc. Natl. Acad. Sci. USA 92:7844-7848 (1995)), swine (Baetscher, M. W. et al., International Patent Application No. WO 95/28412 (1995)), bird (Shuman, R. M., Experientia 47:897-905 (1991)), fish (Wakamatsu, Y. et al., Mol. Mar. Biol. Biotech. 3:185-191 (1994)), guinea pig, cow, dog, horse, cat, goat, sheep, reptile, amphibian, human, and ape.

Primordial germ cell (PGC) derived ES cells are similar to the previously described ES cells in terms of growth properties and uses. In contrast to ES cells, PGC cells are established from primordial germ cells in the germinal ridges of early embryos, rather than from the inner cell mass of blastocysts (Matsui, Y. et al., Cell 70:841-847 (1992)). Cell culturing conditions for establishing and growing PGC-derived ES cell lines require serum (e.g., FBS) and growth factors. The supplement and medium of the present invention can be used to replace the serum component in media used to establish and grow PGC-derived ES cells.

Once an ES cell line has been established, it must be cryopreserved for future use. It is also routine during the gene targeting process to preserve ES clones for reconstitution at a later date. Freezing media generally consist of 5-10% DMSO, 10-90% FBS and 55-85% DMEM media. The supplement of the present invention can be used as a serum substitute for cryopreservation and reconstitution purposes. The conditions for cryopreservation of such cells with the supplement of the invention include 0.5-95% supplement, 1-10% of a cryoprotectant (e.g., dimethylsulfoxide (DMSO)), and 1-90% of a basal medium. ES cells can be frozen under such conditions at about −80° C. and below. ES cells can remain frozen indefinitely at temperatures less than or equal to about −135° C.

When growing or expanding ES cells, inactivated feeder cells are usually prepared by plating feeder cells in DMEM media containing 10% FBS (which does not have to be ES qualified) at least several hours prior to the culturing of ES cells. This time frame allows the feeder cell layer to attach itself and to spread onto the culture dish. Prior to the addition of ES cells and ES cell medium, the medium containing 10% FBS is removed. The medium and supplement of the invention can be used as a substitute for serum containing medium and serum, respectively, for the plating of the fibroblast feeder cells. Preferably, attachment factors are added when using the supplement or the medium of the present invention to grow such feeder cells.

As discussed supra, ES cells are sometimes grown in serum-supplemented medium, together with a growth factor, such as LIF, to prevent the differentiation of ES cells in culture. The invention can be used with or without one or more of such factors, depending on the characteristics of the particular ES cell line.

Some ES cell lines have been isolated in a feeder-free manner or weaned off feeder cells at some point during culturing. Generally, these feeder-free lines are grown on gelatin treated plates in serum-containing medium supplemented with LIF or other growth factors. The supplement of present invention can be used for the growth and maintenance of feeder-free ES lines as a direct substitute for the serum commonly used. Alternatively, the medium of the present invention can be used to culture feeder-free ES lines.

In addition to gene targeting, another way in which ES cell lines find use is as a model system to study cell differentiation. Here, one application is the use of differentiated ES cells as a source of stem cells (e.g., hematopoietic stem cells) that would otherwise be very difficult to obtain (Keller, G. M., Curr. Op. Cell. Biol. 7:862-869 (1995)). In differentiation studies, serum-supplemented medium (with or without additional growth factors) is used to enhance the development of particular cell types. Controlled ES cell differentiation can be facilitated by the present invention. By using a defined growth medium, with or without added, defined factors, rather than a serum-supplemented medium containing undefined factors, the researcher can exert greater control over the differentiation of ES cells in culture. Differentiation can be induced by the addition of a differentiation factor or by changing the culturing conditions to induce ES cells to form one or more particular types of cells.

The supplement or the medium of the present invention can be in liquid form or can be maintained in dry form. Medium ingredients can be dissolved in a liquid carrier or maintained in dry form. The type of liquid carrier and the method used to dissolve the ingredients into solution vary and can be determined by one of ordinary skill in the art with no more than routine experimentation.

The supplement or the medium of the present invention can be made as a concentrated formulation (greater than 1× to 1000×) or as a 1× formulation. Preferably, the solutions comprising ingredients are more concentrated than the concentration of the same ingredients in a 1× media formulation. For example, the ingredients can be 10 fold more concentrated (10× formulation), 25 fold more concentrated (25× formulation), 50 fold more concentrated (50× concentration), or 100 fold more concentrated (100× formulation). In particular, the supplement or the medium of the present invention can be made by dividing the ingredients into compatible, concentrated subgroups. See U.S. Pat. No. 5,474,931.

If the ingredients of the supplement or the medium are prepared as separate concentrated solutions, an appropriate (sufficient) amount of each concentrate is combined with a diluent to produce a less concentrated formulation or a 1× formulation. Typically, the diluent for the subgroups used is water but other solutions including aqueous buffers, aqueous saline solution, or other aqueous solutions may be used according to the invention.

The supplement or the medium or concentrated formulation of the present invention (both aqueous and dry forms) are typically sterilized to prevent unwanted contamination. Sterilization may be accomplished, for example, by ultraviolet light, heat sterilization, irradiaiton, or filtration.

Compounds containing trace element moieties can be prepared in solution. Preferably, compounds containing trace element moieties are grouped in concentrated solutions and stored. For example, it is possible to make 1000-10,000× chemical stock solutions, which can be stored as liquids or frozen in the appropriate aliquot sizes for later use.

The concentration ranges within which ingredients are believed to support the growth of ES and other cells in culture are listed in Tables 1-3. These ingredients can be combined to form the cell culture medium supplement of the present invention. As will be readily apparent to one of ordinary skill in the art, the concentration of a given ingredient can be increased or decreased beyond the range disclosed and the effect of the increased or decreased concentration can be determined using only routine experimentation.

The concentrations of the ingredients of the supplement and of the medium of the present invention are the concentrations listed in Tables 1-3. Table 1 provides the concentrations of non-trace element moiety-containing ingredients. The second column in Table 1 provides ingredient concentrations in the serum-free supplement. The third column in Table 1 provides the range of final ingredient concentrations which can be present in the 1× medium. The fourth column in Table 1 provides the final concentration for each ingredient in a preferred embodiment of the 1× medium.

Table 2 provides the concentrations of trace element moiety ingredients.

The second column in Table 2 provides ingredient concentrations in the serum-free supplement. The third column in Table 2 provides the range of final ingredient concentrations which can be present in the 1× medium. The fourth column in Table 2 provides the final concentration for each ingredient in a preferred embodiment of the 1× medium.

Table 3 provides the concentrations of trace element moiety-containing compounds which can be combined to make the serum-free supplement and the medium of the present invention. The second column in Table 3 provides ingredient concentrations in the serum-free supplement. The third column in Table 3 provides the range of final ingredient concentrations which can be present in the 1× medium. The fourth column in Table 3 provides the final concentration for each ingredient in a preferred embodiment of the 1× medium.

As will be apparent to one of ordinary skill in the art, the trace element moieties may react with ingredients in solution. Thus, the present invention encompasses the formulation disclosed in Tables 1-3 as well as any reaction mixture which forms after the ingredients in Tables 1-3 are combined.

To make the serum-free supplement of the present invention, the amino acids are diluted in cell culture grade water as a 3× concentrate. The pH is adjusted to 0.8 to 1.0 to allow for complete solubilization and to assure stability during storage at 2° to 8° C. Included in this concentrated subgroup is the reduced glutathione and the salt of L-ascorbic acid-2-phosphate (e.g., a Mg-salt). See U.S. Pat. No. 5,474,931. Because ascorbic acid has a relatively short half-life in solution, the phosphate salt is used to enhance the stability of ascorbic acid. The AlbuMAX® I powder is made up as a 3× concentrate in cell culture grade water and allowed to dissolve. If the solution is to be stored, it should be filter sterilized. The present invention also encompasses any substitution for AlbuMax® I, such as other albumins (lipid-free, lipid-poor or lipid-rich) from bovine, human or other sources, and extracts or hydrolysates.

The pH of the amino acid solution is raised to about 7.0-7.4 and then the albumin solution and transferrin are added. Insulin is presolubilized in 0.03 N HCl and the pH is brought up to 10.0 with 0.5 N NaOH. Insulin can also be solubilized at a pH greater than 10 and then added. Insulin is available from both recombinant and animal (including human) sources. In one preferred embodiment, bovine zinc insulin is used.

The trace element moieties are made up as concentrated stock solutions (e.g., 1000×) in 0.01N HCl, which is made in cell culture grade water. After solubilization, the trace element moiety solution can be immediately added to the amino acid solution or can be filtered and stored under nitrogen gas at −70° C.

Transferrin can be iron-poor or iron-saturated and can be from different sources (bovine, human, etc.). In a preferred embodiment, iron-saturated human transferrin is used.

The pH of the albumin-amino acid-transferrin mixture is adjusted with 5N NaOH to pH 7.7 to 7.9 and the insulin and trace are elements added. Cell culture grade water is added to give the desired volume and the solution is filter-sterilized. This supplement can now be used in place of serum and at the same concentration as serum for the growth of ES cells and other cells in culture.

Preferably, the supplement of the present invention is stored at about 4° C. and most preferably at about −20° C., although the supplement may be stored at lower temperatures (e.g., about −80° C.). Preferably, the medium of the present invention is stored at about 4° C.

Various substitutes (e.g., transferrin substitutes, insulin substitutes, albumin substitutes, etc.) can be used to prepare the supplement or the medium of the present invention. The concentrations and procedures for making the supplement or the medium of the present invention with such substitutes can be determined by one of ordinary skill in the art without undue experimentation.

The present invention also provides a eukaryotic cell culture medium prepared by combining a basal medium with the serum-free supplement of the present invention. The combination can be accomplished by mixing or admixing the basal medium with the serum-free supplement. Suitable basal media include, but are not limited to Dulbecco's Modified Eagle's Medium (DMEM), Minimal Essential Medium (MEM), Basal Medium Eagle (BME), RPMI 1640, F-10, F-12, α Minimal Essential Medium (αMEM), Glasgow's Minimal Essential Medium (G-MEM), and Iscove's Modified Dulbecco's Medium.

Preferably, the osmolarity of the 1× medium is between about 280 and 310 mOsmol. However, osmolarity of the 1× medium can be as low as about 260 mOsmol and as high as about 350 mOsmol. Preferably, the basal medium is supplemented with about 2.2 g/L sodium bicarbonate. However, up to about 3.7 g/L sodium bicarbonate can be used. The medium can be further supplemented with L-glutamine (final concentration in the 1× medium is about 2 mM), one or more antibiotics, NEAA (final concentration in the 1× medium is about 100 μM), 2-mercaptoethanol (final concentration in the 1× medium is about 100 μM), and for ES cells, LIF (final concentration in the 1× medium is about 10 ng/mL).

The serum-free supplement and the medium of the present invention can be used to culture ES cells derived from a number of animals, including human, monkey, ape, mouse, rat, hamster, rabbit, guinea pig, cow, swine, dog, horse, cat, goat, sheep, bird, reptile, amphibian, and fish.

The serum-free supplement and the medium of the present invention can also be used to culture other types of cells besides ES cells. For example, BHK 21, VERO, HeLa, Hep2, mouse T-cell lines (e.g., CDC-25), transformed lymphocyte cell lines (e.g., HL6), LLCMK2, PC-12, hybridoma cells, fibroblasts, or other cell lines can be cultured in a basal medium supplemented with the serum-free supplement of the present invention. Preferably, the supplement and the medium of the present invention are used to culture either ES or hybridoma cells. Most preferably, the supplement and the medium of the present invention are used to culture ES cells.

To passage ES cells, the culture is first rinsed once or twice with Ca²⁺, Mg²⁺-free Dulbecco's phosphate buffered saline (DPBS). Sufficient trypsin-EDTA (0.25% trypsin, 1 mM EDTA) is added to just cover the cell layer and the culture vessel is returned to the incubator. After a few minutes, the ES cell colonies and the feeder cells have detached from the plastic vessel and can be further dissociated by pipetting. Growth medium is added to quench trypsin activity and the cells are generally pelleted by centrifugation. The supernatant is removed and the cells are resuspended in fresh growth medium. The cells are transferred to fresh culture vessels containing new feeder layers. The ES cells are not separated from the old feeder cells. The old feeder cells will not attach efficiently in the new culture.

Those of ordinary skill in the art are familiar with methods for culturing ES cells and feeder cells. Guidelines for ES cell culture are outlined in Hogan, G. et al., eds., Manipulating the Mouse Embryo: A Laboratory Manual, Cold Spring Harbor Laboratory Press, Plainview, N.Y. (1994); and Robertson, E. J., ed., Teratocarcinomas and Embryonic Stem Cells: A Practical Approach, IRL Press, Oxford, UK (1987).

Primary mouse embryonic fibroblasts or STO cells are typically used as feeder cells, although other types of fibroblast cells may be used. Primary mouse embryonic fibroblasts are produced by culturing minced, approximately 13 day old embryos and allowing the outgrowth of the fibroblast population over a few passages. In contrast, STO cells are a permanent cell line of embryonic lineage and can be cultured for a more extended time than primary cells. Feeder cells of either type are inactivated by treatment with mitomycin C or gamma irradiation prior to use. While the feeder cells remain metabolically active after such treatment, this treatment renders the feeder cells mitotically inactive. Each time ES cells are passaged they are placed onto a fresh layer of feeder cells.

The present invention also provides a composition comprising ES cells in a serum-free medium, wherein the serum-free medium, which is supplemented with the serum-free supplement of the invention, is capable of supporting the growth of the ES cells in serum-free culture. Aliquots of this composition can be frozen at about −80° C. and below. Aliquots of this composition can be stored indefinitely at less than or equal to about −135° C. After an aliquot of the composition has been thawed and opened, using sterile cell culture technique, the ES cells can be cultivated in serum-free culture. Animals from which ES cells can be obtained include human, monkey, ape, mouse, rat, hamster, rabbit, guinea pig, cow, swine, dog, horse, cat, goat, sheep, bird, reptile, amphibian, and fish.

TABLE 1
CONCENTRATIONS OF NON-TRACE ELEMENT MOIETY
INGREDIENTS

| | A Preferred | | A Preferred |
|---|---|---|---|
| | Embodiment in | Concentration | Embodiment in |
| | Supplement | Range in 1X | 1X Medium |
| | (mg/L) | Medium (mg/L)* | (mg/L)* |
| Ingredient | (About) | (About) | (About) |
| Glycine | 150 | 5-200 | 53 |
| L-Histidine | 940 | 5-250 | 183 |
| L-Isoleucine | 3400 | 5-300 | 615 |
| L-Methionine | 90 | 5-200 | 44 |
| L-Phenylalanine | 1800 | 5-400 | 336 |
| L-Proline | 4000 | 1-1000 | 600 |
| L- | 100 | 1-45 | 15 |
| Hydroxyproline | | | |
| L-Serine | 800 | 1-250 | 162 |
| L-Threomne | 2200 | 10-500 | 425 |
| L-Tryptophan | 440 | 2-110 | 82 |
| L-Tyrosine | 77 | 3-175 | 84 |
| L-Valine | 2400 | 5-500 | 454 |
| Thiamine | 33 | 1-20 | 9 |
| Reduced | 10 | 1-20 | 1.5 |
| glutathione | | | |
| Ascorbic acid-2- | 330 | 1-200 | 50 |
| PO₄ (Mg salt) | | | |
| Transferrin (iron | 55 | 1-50 | 8 |
| sat.) | | | |
| Insulin | 100 | 1-100 | 10 |
| Sodium selenite | .07 | .000001-.0001 | 0.00001 |
| AlbuMAX ® I | 83,000 | 5000-50,000 | 12,500 |

*When used at 15% in DMEM.

TABLE 2
CONCENTRATIONS OF TRACE ELEMENT MOIETIES

| | | | A Preferred |
|---|---|---|---|
| | A Preferred | | Embodiment |
| | Embodiment in | Concentration | in 1X |
| | 1X Supplement | Range in 1X | Medium |
| | (mg/L) | Medium (mg/L) | (mg/L) |
| Ingredient | (About) | (About) | (About) |
| Ag⁺ | 0.0006 | 0.0000006-0.006 | 0.00009 |
| Al³⁺ | 0.0007 | 0.00001-0.001 | 0.0001 |
| Ba²⁺ | 0.008 | 0.00005-0.005 | 0.001 |
| Cd²⁺ | 0.03 | 0.00003-0.03 | 0.005 |
| Co²⁺ | 0.003 | 0.00003-0.003 | 0.0005 |
| Cr³⁺ | 0.0003 | 0.00000008-0.0008 | 0.00004 |
| Ge⁴⁺ | 0.003 | 0.000007-0.0007 | 0.0005 |
| Se⁴⁺ | 0.02 | 0.00005-0.005 | 0.007 |
| Br⁻ | 0.0004 | 0.0000007-0.0007 | 0.00006 |
| I⁻ | 0.0007 | 0.000008-0.0008 | 0.0001 |
| Mn²⁺ | 0.0004 | 0.000003-0.003 | 0.00006 |
| F⁻ | 0.010 | 0.00005-0.005 | 0.002 |
| Si⁴⁺ | 0.01 | 0.0001-0.1 | 0.02 |
| V⁵⁺ | 0.003 | 0.000004-0.004 | 0.0004 |
| Mo⁶⁺ | 0.005 | 0.0000008-0.0008 | 0.0007 |
| Ni²⁺ | 0.0002 | 0.000002-0.0002 | 0.00003 |
| Rb⁺ | 0.005 | 0.0000007-0.007 | 0.0008 |
| Sn²⁺ | 0.0002 | 0.0000006-0.00006 | 0.00003 |
| Zr⁴⁺ | 0.01 | 0.00005-0.005 | 0.0001 |

TABLE 3
CONCENTRATIONS OF TRACE ELEMENT MOIETY-CONTAINING
COMPOUNDS

| | A Preferred | | A Preferred |
|---|---|---|---|
| | Embodiment in | Concentration | Embodiment |
| | Supplement | Range in 1X | in 1X Medium |
| | (mg/L) | Medium (mg/L) | (mg/L) |
| Ingredient | (About) | (About) | (About) |
| AgNO₃ | 0.0009 | 0.000001-0.001 | 0.0001 |
| AlCl₃.6H₂O | 0.006 | 0.0001-0.01 | 0.0009 |
| Ba(C₂H₃O₂)₂ | 0.01 | 0.0001-0.01 | 0.002 |
| CdSO₄.8H₂O | 0.08 | 0.0001-0.1 | 0.01 |
| CoCl₂.6H₂O | 0.01 | 0.0001-0.01 | 0.002 |
| Cr₂(SO₄)₃.1H₂O | 0.003 | 0.000001-0.0001 | 0.0005 |
| GeO₂ | 0.003 | 0.00001-0.001 | 0.0005 |
| Na₂SeO₃ | 0.007 | 0.0001-0.01 | 0.001 |
| H₂SeO₃ | 0.02 | 0.0001-0.01 | 0.002 |
| KBr | 0.0006 | 0.000001-0.0001 | 0.00009 |
| KI | 0.0009 | 0.00001-0.001 | 0.0001 |
| MnCl₂.4H₂O | 0.002 | 0.00001-0.001 | 0.0003 |
| NaF | 0.02 | 0.0001-0.01 | 0.003 |
| Na₂SiO₃.9H₂O | 1 | 0.001-1.0 | 0.2 |
| NaVO₃ | 0.006 | 0.00001-0.01 | 0.0009 |
| (NH₄)₆Mo₇O₂₄.4H₂O | 0.06 | 0.00001-0.01 | 0.009 |
| NiSO₄.6H₂O | 0.001 | 0.00001-0.001 | 0.0002 |
| RbCl | 0.007 | 0.000001-0.01 | 0.001 |
| SnCl₂ | 0.0003 | 0.000001-0.0001 | 0.00005 |
| ZrOCl₂.8H₂O | 0.02 | 0.0001-0.01 | 0.0024 |

The present invention also provides a product of manufacture comprising a container means containing an aliquot of ES cells and the supplement of the invention. The present invention also provides a product of manufacture which is a container means containing an aliquot of the composition of ES cells in the serum-free medium and the serum-free medium of the invention. The present invention also provides a product of manufacture comprising one or more container means, wherein a first container means contains the supplement of the invention or the serum-free medium of the invention. Optionally, a second container means contains a basal medium. Optionally, a third container means contains ES cells. Preferably, the products of manufacture containing the supplement of the invention are stored at about 4° C. and preferably at about −20° C. Products of manufacture containing the medium of the invention are preferably stored at about 4° C.

The present invention also provides a method of expanding ES cells in serum-free culture. In this method, ES cells are cultivated in serum-free culture using a serum-free medium of the present invention. This serum-free medium contains the serum-free supplement of the present invention.

The present invention also provides a method of controlling or preventing the differentiation of ES cells in serum-free culture. Because the supplement of the present invention is serum-free, it facilitates maintenance of the undifferentiated, pluripotent state of ES cells in culture. If desired, the cell culture medium can be supplemented with leukemia inhibitory factor (LIF) (Life Technologies, Inc.). Other factors which inhibit ES cell differentiation include but are not limited to steel factor (Matsui, Y. et al., Cell 70:841-847 (1992)); and ciliary neurotrophic factor (CNTF) (Conover, J. C. et al., Development 119:559-565 (1993)), and oncostatin M (Conover, J. C. et al., Development 119:559-565 (1993)).

Differentiation of ES cells can be assessed using an alkaline phosphatase histochemical assay (Pease, S. et al., Devel. Biol. 141:344-352 (1990)). For example, Sigma diagnostic kit 86-R (Sigma Chemical, St. Louis, Mo.), can be used, as illustrated in Example 1. Other markers can be used to assess degree of ES cell differentiation. For example, ECMA-7 or TROMA-1 monoclonal antibodies can be used (Brulet, P. et al., Proc. Natl. Acad. Sci. USA: 77:4113-4117 (1980)). Thus, one of ordinary skill can, by cultivating ES cells in serum-free culture using the serum-free supplement, expand ES cells and prevent them from differentiating in culture.

The serum-free supplement of the present invention can also be used to cause ES cells to differentiate into a cell type of interest. Those of ordinary skill in the art are familiar with techniques for differentiating ES cells in vitro. For example, see Dinsmore, J. et al., Cell Transplantation 5:131-143 (1996); Ray, W. J., et al., J. Cell. Physiol. 168:264-275 (1996); Palacios, R. et al., Proc. Natl. Acad. Sci. USA 92:7530-7534 (1995); Setlow, J. K., Genetic Engineering: Principles and Methods 16:17-31, Plenum Press (1994); Pedersen, R. A., Reprod. Fertil. Dev. 6:5543-552 (1994); Doetschman, T. et al., Hypertension 22:618-6629 (1993); Snodgrass, H. R. et al., J. Cell. Biochem. 49:225-230 (1992); and Hollands, P., Human Reprod. 6:79-84 (1991).

In this embodiment, ES cells are expanded in serum-free culture comprising a basal medium supplemented with the serum-free supplement of the present invention. Differentiation is inhibited during expansion. Undifferentiated ES cell colonies are removed from the culture vessel, transferred to a new culture vessel, and cultivated in the serum-free medium of the present invention in specific ways to form a population of the differentiated cell type. Alternatively, the ES cells are treated with one or more growth factors which will cause the ES cells to differentiate into the cell type of interest.

In order to facilitate differentiation, the cultured ES cells can be treated with one or more nucleic acid constructs, wherein each construct contains a nucleic acid molecule which encodes a protein of interest, the expression of which will contribute to the differentiation of the ES cell into the cell type of interest.

Cell types into which ES cells can be forced to differentiate include, but are not limited to, neurons, myocardial atrial cells, myocardial ventricular cells, skeletal muscle, glial cells, endothelial cells, epithelial cells, kidney cells, liver cells, and hematopoietic cells (including hematopoietic stem, progenitor, and precursor cells, leukocytes, macrophages, eosinophils, neutrophils, red blood cells, reticulocytes, B cells, and T cells).

ES cells can be incubated with specific factors in order to induce differentiation of the ES cells into a particular type of cell. Such factors are well know to those of ordinary skill in the art. For example, such factors include, but are not limited to, interleukins, cytokines, colony stimulating factors, growth factors, and interferons.

The serum-free supplement of the present invention can also be used to prepare a cell type of interest for explantation into a mammal. In this embodiment, cells which have been caused to differentiate (supra) are introduced into a mammal. For example, ES cells which have been caused to differentiate into a hematopoietic stem, precursor, or progenitor cell can be introduced into the bone marrow or the bloodstream of the mammal. Any differentiated cell type can be introduced into the bloodstream or bone marrow of the mammal. Alternatively, the differentiated cell type of interest can be introduced into a tissue, such as skin, brain, skeletal muscle, heart, lung, kidney, bladder, breast, stomach, esophagus, small intestine, large intestine, testicle, prostate gland, uterus, ovary, lymph gland, liver, spleen, thymus, and thyroid gland. Mammals into which a differentiated cell can be explanted include human, monkey, ape, mouse, rat, hamster, rabbit, guinea pig, cow, swine, dog, horse, cat, goat, and sheep.

The serum-free supplement of the present invention can also be used to express a recombinant protein in ES cells (or other cell types) cultivated in serum-free culture. Generally, recombinant protein is obtained by isolating ES cells from cultured blastocysts, and cultivating the isolated ES cells in serum-free culture under conditions suitable to facilitate ES cell expansion and prevent ES cell differentiation. More specifically, recombinant protein is obtained by introducing a nucleic acid construct (i.e., DNA), comprising a nucleic acid molecule which encodes a protein of interest into ES cells (e.g., by electroporation or by transfection methods known by those of ordinary skill in the art). After the nucleic acid construct has been introduced, recombinant ES cells are selected and cultivated in serum-free culture comprising a basal medium supplemented with the serum-free supplement of the present invention. Recombinant protein can be isolated from ES cells by methods well known to those of ordinary skill in the art. For example, see Ausubel, F. M. et al., eds., Current Protocols in Molecular Biology, John Wiley & Sons (1994). If the ES cells are cocultivated with feeder cells, the recombinant protein can be isolated from the mixture of ES cells and feeder cells. If the recombinant protein is secreted by the ES cells, the recombinant protein can be harvested from the serum-free medium in which ES cells are cultivated.

The serum-free supplement of the present invention can also be used to produce a transgenic animal. This is accomplished by cultivating ES cells in serum-free culture, introducing a nucleic acid molecule into ES cells, selecting a recombinant ES cell clone, expanding the recombinant ES cell clone to form a population, injecting an aliquot of the recombinant ES cell clonal population into a blastocyst, transferring the injected blastocyst into a host pseudopregnant female animal, and selecting transgenic offspring. The present invention also provides a transgenic animal obtained by this method.

A transgenic animal can also be produced by cultivating ES cells in serum-free culture, introducing a nucleic acid molecule into ES cells, selecting a recombinant ES cell clone, expanding the recombinant ES cell clone to form a population, co-culturing a small number of the ES cells with early stage embryos (e.g., eight cell morulae) to form aggregates, transferring the aggregated embryos into a host pseudopregnant female animal, and selecting transgenic offspring. The present invention also provides a transgenic animal obtained by this method.

Animals which can be used to produce a transgenic animal include human, monkey, ape, mouse, rat, hamster, rabbit, guinea pig, cow, swine, dog, horse, cat, goat, sheep, bird, reptile, amphibian, and fish. The transgenic manipulation accomplished can be any transgenic manipulation including, but not limited to, a gain of function alteration, including a dominant positive augmentation or a targeted correction (Merlino, G. T., FASEB J. 5:2996-3001 (1991)); and a loss of function alteration, including a dominant negative interference, a targeted knockout, or a conditional knockout (Merlino, G. T., FASEB J. 5:2996-3001 (1991); Barinaga, M., Science 265:26-28 (1994); Gu, H. et al., Science 265:103-106 (1994)). This method can be practiced routinely by those of ordinary skill in the art.

The serum-free supplement or medium of the present invention can be used to produce recombinant protein from a transgenic animal. In this embodiment, ES cells used to produce the transgenic animal are cultivated in serum-free culture which comprises a basal medium supplemented with the serum-free supplement of the present invention. In this embodiment, the transgene may be operably linked to a tissue-specific promoter. See U.S. Pat. No. 5,322,775. The recombinant protein is isolated from the blood or the milk of the transgenic animal. Animals which can be used to practice this embodiment include cows, sheep, goats, mice, rabbits, etc.

The serum-free supplement of the present invention can also be used to isolate ES cells from an animal. Such isolated ES cells can be used to establish new and useful lines of ES cells. In this embodiment, isolated ES cells are cultivated in serum-free culture comprising a basal medium supplemented with the serum-free supplement of the present invention. Animals from which ES cells can be obtained using the supplement and the medium of the present invention include human, monkey, ape, mouse, rat, hamster, rabbit, guinea pig, cow, swine, dog, horse, cat, goat, sheep, bird, reptile, amphibian, and fish.

Having now fully described the present invention, the same will be more clearly understood by reference to certain specific examples which are included herewith for purposes of illustration only, and are not intended to be limiting of the invention.

In the examples that follow, unless otherwise specified, all media, media supplements, growth factors and cell culture reagents were produced by Life Technologies, Inc. (Gaithersburg, Md.). Feeder cell medium was composed of DMEM (cat # 11965) with final ingredient concentrations as follows: 10% FBS, 2 mM L-glutamine, 50 U/mL penicillin and 50 μg/mL streptomycin.

In the examples that follow, ES cell serum-supplemented medium was composed of DMEM with final concentrations of 15% ES qualified FBS, 2 mM L-glutamine, 100 μM NEAA, 50 U/mL penicillin, 50 μg/mL streptomycin and 100 μM 2-mercaptoethanol (Sigma). If LIF was used in the ES cell medium, ESGRO™ (murine recombinant LIF) was added in order to obtain a final concentration of 1000 U/mL (10 ng/mL).

### EXAMPLE 1

### Establishment of the Basic Formulation

ES D3 ES cells were used (Doetschman, T. C. et al., J. Embryol. Exp. Morph. 87:27-45 (1985)). Unless otherwise specified, D3 cells at passage 15 were used. Trypsin-EDTA (0.25%, 1 mM) was used to remove cells from plates after rinsing the cell layer with phosphate buffered saline (PBS). Cells were cultured in a humidified 37° C., 10% CO₂ incubator.

The protocol for a media formulation evaluation assay was as follows. The source of ES cells for the experiments was a sub-confluent dish of ES cells maintained on a feeder layer in ES cell medium with LIF. Feeder layers for experimental conditions were established in 6 well plates (NUNC) by seeding 3-5×10⁴ feeder cell/cm² and allowing the cells to attach. ES cells were trypsinized to form a cell suspension. Trypsin activity was quenched with serum-supplemented medium, and cells were pelleted by centrifugation at 500× g. The medium was removed and the ES cells were resuspended in DMEM containing 2 mM L-glutamine, 50 U/mL penicillin, 50 ug/mL streptomycin, 100 uM NEAA, and 100 uM 2-mercaptoethanol (final concentrations).

ES cells were then mixed with respective test media (described infra) at a concentration of 90 cells/iL. Feeder cell media was then removed from the feeder plates, and the feeder layers were washed once with 2 mL of DMEM (that was not supplemented with serum or any other additives). 2.5 mL of test medium and ES cells (225/well) were added to each well of feeder cells. Test conditions were assayed in triplicate (3 wells/test condition). The cells were incubated for 7 days while observations were made regarding ES cell growth parameters. Incubation conditions were 37° C., 10% CO₂ in air, and humidified atmosphere.

At the end of the 7 day culture period, observations were made and then ES cells were harvested, fixed and assayed for the presence of alkaline phosphatase by using a histochemical assay (Sigma diagnostic kit 86-R, Sigma, St. Louis, Mo.). Cells were fixed and assayed according to the manufacturer's directions. In this assay, cells which express alkaline phosphatase stain dark pink or red. ES cell colonies were rated in terms of morphology and strength of alkaline phosphatase staining according to the following parameters. Class I colonies are round, stain dark pink, and have the desired, undifferentiated colony morphology characterized by a well-defined colony border. Class II colonies are those that have begun to differentiate, are stained at least 60% pink, and have a more flattened appearance, with a poorly defined border. Class III colonies demonstrate clear signs of colony differentiation, with very little to no pink stain and a flattened appearance with poor border definition. Plating efficiency was determined by dividing the total number of colonies obtained by the input number of ES cells (225/well).

A serum-free medium supplement was tested in an evaluation assay, as described above, for its ability to promote the growth and maintenance of undifferentiated ES cells. The basic formulation of this supplement was as described in Tables 1 and 3 (far right column of each table), but without the L-ascorbic acid-2-phosphate. This formulation was tested in conjunction with some alternate serum-free formulations containing components known to be beneficial for other cell types. These other components, 15 μg/L ferric citrate, 0.3 μg/L glycl-histidyl-lysine and 300μg/L ethanolamine, were tested in all combinations in a +/−fashion. The formulations were added to DMEM to a final concentration of 15%. In all cases, the general plating efficiency and number of undifferentiated ES colonies observed were no different with or without these components. Thus, it was concluded that ferric citrate, glycl-histidyl-lysine, and ethanolamine are not required for optimal ES cell growth.

### EXAMPLE 2

### Improvement to the Basic Formulation

The formulation that performed the best in Example 1 was then further evaluated to see whether improvements could be made to enhance its performance. This formulation was the same as the formulation in the far right column of each of Tables 1 and 3, except that no ascorbic acid phosphate was present.

One aspect of the supplement that was sub-optimal related to the morphology of the feeder layer in ES cell cultures maintained in the same culture vessel for more than three days. Generally, ES cells are passaged every two to three days. Typically, during selection of antibiotic-resistant ES cells, cultures are maintained without passaging for ten or more days. However, during these extended culture periods in medium supplemented with the supplement of the present invention (without ascorbic acid-2-phosphate), the feeder layer was noted to become sparse and patchy due to the detachment of individual feeder cells. The detached cells were seen floating in the growth medium. Further, the attached remaining feeder cells exhibited an undesirable morphology (i.e., spindly morphology, ragged outlines), in comparison to control cells grown in medium supplemented with FBS. In addition, ES cell colonies growing on these spindly, ragged-looking feeder cells were noticeably reduced in size overall, in comparison to ES cell colonies grown in medium supplemented with FBS.

In order to improve the formulation, the addition of L-ascorbic acid-2-phosphate to the formulation was evaluated. In an evaluation assay (as in Example 1), the medium was supplemented with the serum-free supplement (to a fmal concentration of 15%), either with or without L-ascorbic acid-2-phosphate (50 mg/L final concentration), and 10 ng/mL LIF (final concentration).

The averaged results of three wells are shown in Table 4. In Table 4, numbers outside of parenthesis are the number of ES cell colonies which displayed the indicated degree of differentiation. The numbers within parentheses indicate what percentage of total ES cell colonies that the colonies with the indicated degree of differentiation represented. In Table 4, “good” feeder cell morphology reflects a more fibroblast-like character and smooth borders, rather than a spindly, ragged-looking character.

The results in Table 4 indicate that L-ascorbic acid-2-phosphate directly improved the appearance of the feeder layer independent of the generally beneficially action of LIF in the growth media. With LIF in the culture media, L-ascorbic acid-2-phosphate had virtually no effect on the morphology class of colonies obtained. However, L-ascorbic acid-2-phosphate did increase average colony size (an indication of growth rate) somewhat. This was probably due to the improvement of the feeder layer.

Without LIF in the media, the effects were more dramatic. In the absence of LIF, and in the presence of L-ascorbic acid-2-phosphate, the percent of class I colonies was increased, the percent of class III colonies was decreased, and colony size was much improved. In this experiment, while LIF alone had a positive effect on plating efficiency, L-ascorbic acid-2-phosphate alone had little effect on plating efficiency. Since L-ascorbic acid-2-phosphate caused no significant negative effects and led to definite improvements in colony size and feeder layer morphology, L-ascorbic acid-2-phosphate was added to the formulation of the invention.

TABLE 4
Effects of AAP +/− LIF on ES Cells and Feeder Layer

| Experimental | | Class II | Class III | Total Colonies | Colony | Feeder Cell |
|---|---|---|---|---|---|---|
| Condition | Class I (%) | (%) | (%) | (% plating) | Size | Morphology |
| +AAP/+LIF | 167 (98.5%) | 2 (1%) | 1 (0.5%) | 170 (75%) | excellent | good |
| +AAP/−LIF | 27 (28%) | 28 (29%) | 41 (43%) | 96 (42%) | good | good |
| −AAP/+LIF | 158 (98%) | 2 (1.4%) | 1 (0.6%) | 161 (72%) | moderate | moderate |
| −AAP/−LIF | 16 (15%) | 33 (30.5%) | 59 (54.5%) | 108 (48%) | very | poor |
| | | | | | small | |

### EXAMPLE 3

### Routine Growth and Maintenance of ES Cells in the Invention

ES cells were grown and passaged, according to standard ES culture practices known to those of ordinary skill in the art (supra), in DMEM supplemented with LIF (10 ng/mL final concentration) and either the supplement of the present invention (at 15% final concentration) or with ES qualified FBS (at 15% final concentration)

Cultures were maintained for four passages. Cell count and cell morphology were evaluated at each passage. ES cell morphology improved within two days of growth in medium supplemented with the serum-free supplement of the present invention. Over time, the morphology of ES cells cultured in medium supplemented with the serum-free supplement continued to be superior to that of ES cells grown in FBS-supplemented medium. For cells grown in medium supplemented with the serum-free supplement of the present invention, cell counts were at least equal to, if not higher than, cells grown in FBS-supplemented medium. The observed increase in cell count was most likely due to the increased plating efficiency seen with cells cultured in medium supplemented with the serum-free supplement.

After the fourth passage, a chromosome analysis was performed, using the Mouse Y.ES™ system (Life Technologies, Inc.), on cells grown in FBS-supplemented medium and on cells grown in medium supplemented with the serum-free supplement of the present invention. No significant differences were observed between the two sets of cells. All spreads analyzed (25 for each set of cells) showed >90% normal diploid number. Maintenance of normal ploidy and the undifferentiated nature of the ES cells indicate that the culture conditions are suitable for ES cells.

### EXAMPLE 4

### Culture of Other ES Cell Lines in Medium Supplemented with the Serum-free Medium

In order to determine whether the supplement of the present invention is useful for other ES cell lines besides the D3 line, three additional ES lines were cultured in medium supplemented with the serum-free supplement of the present invention. Two mouse strain 129 ES lines, E14 (Hooper, M., Nature 326:292-295 (1987)) and R1 (Nagy, A. et al., Proc. Natl. Acad. Sci. USA 90:8424-8428 (1993)), were evaluated. In addition, a non-129 ES line, TT2 (C57B1/6 X CBA F₁) (Yagi, T. et al., Analyt. Bioch. 214:70-76 (1993)), was evaluated. For all three ES cell lines, cells grown in medium supplemented with the serum-free supplement exhibited a generally improved cell morphology (i.e., rounded cells with smooth cell borders), and less differentiation, in comparison to cells grown in FBS-supplemented medium. Thus, the serum-free supplement of the present invention can be used to cultivate any ES cell line under serum-free conditions.

### EXAMPLE 5

### Comparison of the Serum-free Supplement to ES Qualified FBS and Other Commercially Available Fetal Bovine Sera

An evaluation assay was performed, as in Example 1, in which D3 ES cells were cultured under eight different test conditions. Cells were cultured in media supplemented separately with a) two different manufactured lots of the serum-free supplement of the present invention, b) a lot of ES qualified FBS and c-g) media supplemented with five different lots of commercially available serum (Hyclone, Logan, Utah). In all test conditions, media contained 10 ng/mL LIF (final concentration). The results (average of three wells) are shown in Table 5. In Table 5, numbers outside of parenthesis are the number of ES cell colonies which displayed the indicated degree of differentiation. The numbers within parentheses indicate what percentage of total ES cell colonies that the colonies with the indicated degree of differentiation represented.

The two lots of the serum-free supplement of the present invention performed quite similarly. That is, ES cells exhibited high plating efficiency, almost no differentiation, and excellent cell and colony morphology. The equal performance of the two lots supports the fact that, due to its defined and reproducible composition, pretesting of a given lot of the serum-free supplement for use with ES cell cultures is not necessary.

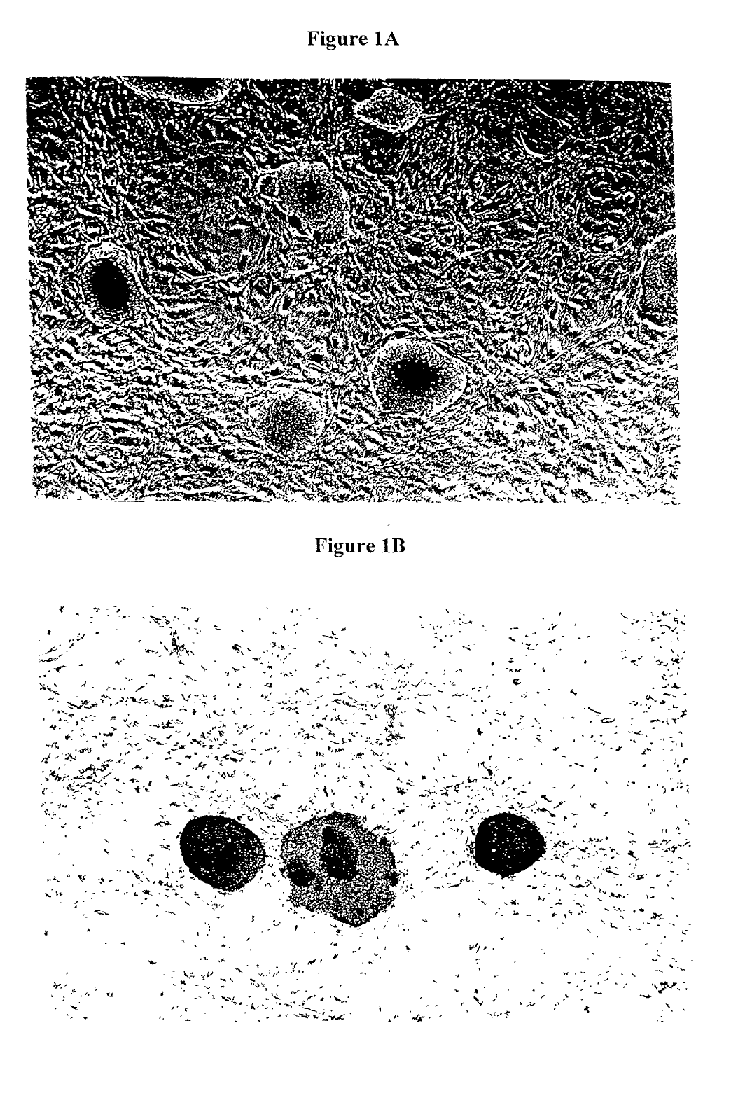

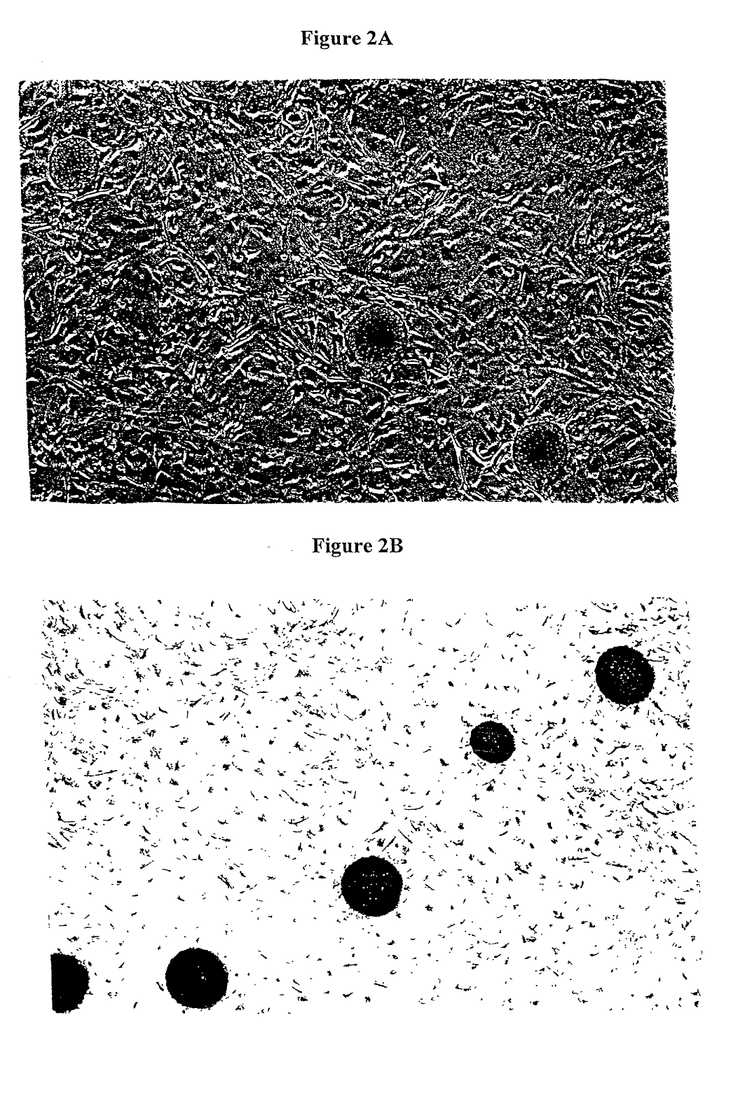

The serum-free supplement is clearly superior to ES qualified FBS (Table 5). The serum-free supplement facilitated increased plating efficiency and resulted in a >50% increase in the number of undifferentiated ES cell colonies. Examples of the excellent morphology and deep staining for alkaline phosphatase found in ES cells grown in the serum-free supplement are shown in FIGS. 1 and 2.

Even more dramatic were the results obtained using the serum-free supplement compared to the five lots of commercially available FBS (Table 5). The results obtained using the commercially available FBS were quite variable lot-to-lot. These results clearly illustrate that FBS must be pre-screened prior to use in ES cell culture. The requirement for pre-screening serum is obviated by the serum-free supplement of the present invention.

### EXAMPLE 6

### Differentiation of ES Cells

When cultivated in serum-supplemented medium, ES cells undergo differentiation in vitro and acquire the morphology and hallmarks of other cell types. By following specific protocols, certain types of differentiated cells can be reproducibly obtained using a differentiation assay (Doetschman, T. C. et al., J. Embryol. Exp. Morph. 87:27-45 (1985)). Briefly, a plate of ES cells was trypsinized and replated, in the absence of feeder cells and in the absence of LIF, onto non-electrostatically charged plastic.

TABLE 5
Results of Comparative Assay

| | Type I | Class II | Class III | Total | |
|---|---|---|---|---|---|
| Test | Colonies | Colonies | Colonies | Colonies | |
| Condition | (%) | (%) | (%) | (% plating) | Colony Characteristics |
| Invention | 227 (99%) | 3 (1%) | 0 | 230 (102%) | round, dark pink colonies |
| Lot A | | | | | with well-defined borders |
| Invention | 215 (99%) | 3 (1%) | 0 | 218 (97%) | round, dark pink colonies |
| Lot B | | | | | with well-defined borders |
| ES Qualified | 140 (73%) | 47 (24%) | 6 (3%) | 193 (86%) | mixture of round, dark pink |
| FBS Control | | | | | colonies and flattened pink |
| | | | | | colonies undergoing |
| | | | | | differentiation |
| Hyclone A | 109 (67%) | 37 (23%) | 16 (10%) | 162 (72%) | varying degrees of colony |
| | | | | | differentiation staining, no |
| | | | | | uniform shape |
| Hyclone B | 104 (69%) | 37 (25%) | 10 (6%) | 151 (67%) | varying degrees of colony |
| | | | | | differentiation staining, no |
| | | | | | uniform shape |
| Hyclone C | 98 (70%) | 34 (24%) | 8 (6%) | 140 (62%) | varying degrees of colony |
| | | | | | differentiation staining, no |
| | | | | | uniform shape |
| Hyclone D | 87 (66%) | 35 (27%) | 10 (7%) | 132 (59%) | varying degrees of colony |
| | | | | | differentiation staining, no |
| | | | | | uniform shape |
| Hyclone E | 95 (72%) | 27 (21%) | 9 (7/%) | 131 (58%) | varying degrees of colony |
| | | | | | differentiation staining, no |
| | | | | | uniform shape |

This allowed the ES cells to aggregate into floating balls in the medium. These balls of cells, called embryoid bodies, began to differentiate. The embryoid bodies were allowed either to continue to grow in suspension culture, or were caused to attach to electrostatically charged plastic (without feeder cells). From embryoid bodies that were attached to plastic, cells grew out from the differentiated mass. A number of various cell types grew out from the embryoid body, including cardiac cells that pulsated in vitro.

When the differentiation assay was performed with ES cells cultured in the serum-free supplement of the invention, the number of embryoid bodies that formed was reduced, relative to cells cultured in FBS-supplemented medium. After extended culture periods (about three weeks), those embryoid bodies that formed in medium supplemented with the serum-free supplement had a much more pronounced, rounded shape. When plated on electrostatically charged plastic and allowed to attach, the embryoid bodies would not attach without the addition of 1% FBS to supply undefined attachment factors. Once attached, the differentiated cells that grew out of the embryoid bodies were quite different than those seen in FBS-supplemented medium. Cells which grew out of differentiated, attached embryoid bodies included those that formed large tubule structures and sacs. In contrast, ES cells cultured in medium supplemented with serum (1% final concentration FBS) did not survive or form embryoid bodies. It is expected that purified attachment factors can be substituted for the 1% serum that was used to supply such factors.

In culture systems in which differentiation of ES cells into various precursor or other differentiated cell types is desirable, using a serum-free growth substance to which specific factors can be added will allow greater experimental control and flexibility.

### EXAMPLE 7

### Selection of G418 resistant ES Cells

The serum-free supplement of the present invention facilitates selection of drug-resistant ES cells. ES cells were grown in either FBS-supplemented medium or in medium supplemented with the serum-free supplement of the present invention. For each set of cells, 3.4×10⁶ cells were subjected to electroporation (in phosphate-buffered saline) with a DNA vector containing the neo gene, which confers resistance to the antibiotic G418.

After electroporation, cells were replated onto neo resistant feeder cells, in either FBS-supplemented medium or medium supplemented with the serum-free supplement of the present invention. Both sets of cells were cultured for 24 hours prior to the addition of the respectively supplemented media and G418. Drug selection was performed, in triplicate plates, at 0, 150, 250, 350 and 450 μg/mL G418 (Geneticin®, Life Technologies, Inc.). ES cells cultured in the absence of G418 were confluent and overgrown in two days. Cultures of drug-free ES cells were terminated at that time.

Colonies of G418-resistant cells were obtained more quickly from cells cultured in medium supplemented with the serum-free supplement of the present invention (i.e., after four days), compared to resistant colonies obtained from cells cultured in FBS-supplemented medium (i.e., six days). Moreover, additional numbers of more resistant colonies were obtained from cells cultured in medium supplemented with the serum-free supplement of the present invention.

The serum-free supplement facilitated better selection of G418-resistant colonies over the entire range of G418 concentrations tested (150 μg/mL-450 μg/mL). For example, at 250 μg/mL G418, a total of 72 resistant colonies were obtained in FBS-supplemented medium (out of the 3.4×10⁶ cells electroporated). In contrast, in cells cultured in medium supplemented with the serum-free medium, 1104 resistant cells were isolated (out of the 3.4×10⁶ cells electroporated). Moreover, these resistant colonies displayed improved morphology (i.e., rounder cells, smooth borders, less differentiated), in comparison to drug-resistant colonies selected in FBS-supplemented medium. It is possible that the increased selection efficiency is due to an increase in the actual efficiency of transformation of ES cells cultured in medium supplemented with the serum-free supplement. Alternatively, it is possible that the increase in level of cell survival conferred by the serum-free supplement contributes to the overall increase in the number of resistant colonies.

### EXAMPLE 8

### Demonstration of the Germline Competence of ES Cells Cultured in Serum-free Supplemented Medium

R1 ES cells (Nagy, A. et al., Proc. Natl. Acad. Sci. USA 90:8424-8428 (1993) at passage 16 were cultured in either FBS-supplemented medium (final concentration 17.5%) or medium supplemented with the serum-free supplement of the present invention (final concentration 17.5%) for 12-14 days (4-5 passages). During the course of this experiment, ES cell colonies grown in the medium supplemented with the serum-free supplement were observed to be rounder and cleaner looking (i.e., exhibited smooth cell borders) than ES cell colonies grown in serum-supplemented medium.

On days 12 or 13 (at passage 20) and day 14 (at passage 21), ES cells cultured in medium supplemented with the serum-free supplement were injected into blastocysts. ES cells cultured in FBS-supplemented medium were injected on day 12 (at passage 20) and 14 (at passage 21). C57B1/6 blastocyts were injected in medium supplemented with either 5% serum-free supplement or with 5% FBS. All injected blastocysts were transferred to host females.

Table 6 shows birth data: total number of mice born, the number of chimeras born, and the sex of the chimeras. In Table 6, numbers outside of parenthesis are the number of pups obtained using the indicated media. The numbers within parentheses indicate what percentage of total animals the indicated category of animals represented. The litter was 70% male, which probably reflects sex conversion of female embryos by the male ES cell line. No significant differences were seen in the % of total pups born or in the % of chimeric pups in the two test conditions. Possible differences in the sex of the chimeric pups could not be adequately judged due to the small number of control pups available for analysis. Overall, excellent germline transmission was obtained. Transmission of the ES cell component was observed in 7 of the chimeras (78%), from both male and female animals. with coat color contributions ranging from 5-100% (Table 7). All offspring appeared to be healthy.

One feature of the present invention was revealed while injecting ES cells, cultured using medium supplemented with the serum-free supplement, into blastocysts. The process of injection of the ES cells into blastocysts requires exacting skills and a high level of technical training. While the injection medium formulation differs slightly from lab to lab, it generally contains at least 5% FBS to ensure that the ES cells remain healthy during the injection process. The injection process is hampered by the inherent stickiness of ES cells cultured in the FBS-supplemented media. The injection pipette becomes easily clogged and requires frequent changing. In contrast, injection medium prepared with the serum-free supplement of the present invention facilitated the formation of ES cell suspensions that were markedly less sticky than the ES cell suspensions obtained using FBS-supplemented medium. Accordingly, the typically technically challenging injection process was rendered easier and less time-consuming.

TABLE 6
Birth Data

| | | Serum-Supplemented |
|---|---|---|
| | Serum-Free Medium | Medium |
| # of Blastocysts Injected | 104 | 32 |
| Live Pups (%) | 20 (19%) | 7 (22%) |
| Chimeric Pups (%) | 10 (50%) | 3 (43%) |
| Male Chimeras (%) | 7 (70%) | 1 (33%) |
| Female Chimeras (%) | 3 (30%) | 2 (67%) |

TABLE 7
Germline Transmission Data

| | |
|---|---|
| Mice Derived from Invention ES Cells | Mice derived from FBS ES Cells |

| Founder | | % agouti coat | | # agouti | Founder | % agouti coat | | # agouti pups/total |
|---|---|---|---|---|---|---|---|---|
| # and sex | | color | Germline | pups/total (%) | # and sex | color | Germline | (%) |
| 1 | male | 100 | yes | 12/35 (34%) | 1 male | 100 | yes | 6/6 (100%) |
| 2 | male | 100 | yes | 22/29 (76%) | 2 female | 70 | no | no pups |
| 3 | male | 100 | yes | 26/29 (90%) | 3 female | 40 | no | 0/17 (0%) |
| 4 | male | 95 | no | 0/30 (0%) | | | | |
| 5 | male | 75 | yes | 5/28 (18%) | | | | |
| 6 | male | 40 | yes | 1/28 (4%) | | | | |
| 7 | male | 5 | yes | 28/29 (96%) | | | | |
| 8 | female | 80 | yes | 7/7 (100%) | | | | |
| 9 | female | 70 | no | 0/6 (0%) | | | | |
| 10 | female | 50 | not bred | — | | | | |

### EXAMPLE 9

### Hybridoma Cell Culture

The serum-free supplement of the present invention can also be used to grow hybridoma cells. Tables 8 and 9 show the results of culturing SP2/0 (Table 8) and AE-1 (Table 9) hybridoma cells. In both Tables 8 and 9, results are presented as the number of cells (×10⁶) per 25 cm² plastic flask (cell culture grade) over four subcultures at 3 to 4 day intervals.

No attachment factors were required. Nor was treatment of the plastic growth surface required. Cells were removed from flasks using standard cell culture techniques. The surface of the culture was washed with cold Dulbecco's phosphate buffered saline (DPBS). This washing was followed by treatment with 1.0 mL of cold trypsin-EDTA (0.25% trypsin, 1 mM EDTA) (Life Technologies, Inc.). The trypsin-EDTA was allowed to sit on the cell surface for three to five minutes and the cells were then detached from the surface of the flask by vigorous agitation against the palm of the hand. Trypsin activity was quenched by the addition of 1.5 mL of soybean trypsin inhibitor (0.1 mg/mL) (Sigma, Cat. No. T9218) in DPBS. The cells were counted using the tr-pan blue exclusion method.

New cultures were plated at 2.5×10⁵ per 25 cm² flask. Plated cells were cultured at 37° C. in a 5% CO₂ atmosphere. Results depicted in both Tables 8 and 9 were obtained in experiments using RPMI 1640 medium supplemented with 2 mM L-glutamine (Life technologies, Inc.).

The results in Tables 8 and 9 show that hybridoma cells can be cultured in the basal medium supplemented with the serum-free supplement of the present invention.

TABLE 8
Growth of Hybridoma Cell Line SP2/0*

Subculture

| Medium | 1 | 2 | 3 | 4 | Mean |
|---|---|---|---|---|---|
| RPMI 1640 | 8.6 | 4.9 | 1.8 | 3.3 | 4.6 |
| 5% FBS | | | | | |
| Serum-free | 9.5 | 6.0 | 1.0 | 5.7 | 5.6 |
| Formulation | | | | | |

*×10⁶ cells/25 cm² flask

TABLE 9
Growth of Hybridoma Cell Line AE-1*

Subculture

| Medium | 1 | 2 | 3 | 4 | Mean |
|---|---|---|---|---|---|
| RPMI 1640 | 11.0 | 7.3 | 5.2 | 5.8 | 7.3 |
| 5% FBS | | | | | |
| Serum-free | 10.8 | 5.5 | 4.9 | 6.3 | 6.9 |
| Formulation | | | | | |

*×10⁶ cells/25 cm² flask

The supplement and the medium of the present invention can be used to culture any hybridoma line. Those of ordinary skill in the art are familiar with other hybridoma lines besides SP2/0 and AE-1. For example, see the American Type Culture Collection Cell Lines and Hybridomas catalog.

All publications, Patent applications, and Patents are herein incorporated by reference to the same extent as if each individual publication, Patent application, or Patent was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A serum-free, eukaryotic cell culture medium supplement comprising one or more ingredients selected from the group consisting of albumins or albumin substitutes, one or more amino acids, one or more vitamins, one or more transferrins or transferrin substitutes, one or more antioxidants, one or more insulins or insulin substitutes, one or more collagen precursors, and one or more trace elements,
wherein a basal cell culture medium supplemented with said supplement is capable of supporting the growth of embryonic stem cells in serum-free culture.

2. A serum-free, eukaryotic cell culture medium supplement comprising an albumin or an albumin substitute and one or more ingredients selected from group consisting of one or more amino acids, one or more vitamins, one or more transferrins or transferrin substitutes, one or more antioxidants. one or more insulins or insulin substitutes, one or more collagen precursors, and one or more trace elements,
wherein a basal cell culture medium supplemented with said supplement is capable of supporting the growth of embryonic stem cells in serum-free culture.

3. The serum-free, eukaryotic cell culture medium supplement according to claim 1, wherein said antioxidant is selected from the group consisting of reduced glutathione and ascorbic acid an ascorbic acid-2-phosphate.

4. The serum-free, eukaryotic cell culture medium supplement according to claim 1, wherein said collagen precursor is selected from the group consisting of L-proline and multimers or derivatives thereof, L-hydroxyproline multimers or derivatives thereof, and ascorbic acid and multimers thereof.

5. The serum-free, eukaryotic cell culture medium supplement according to claim 1, wherein said transferrin substitute is an iron chelate selected from the group consisting of a ferric citrate chelate and a ferrous sulfate chelate.

6. The serum-free, eukaryotic cell culture medium supplement according to claim 5, wherein said transferrin substitute is ferrous sulphate.7 water-EDTA.

7. The serum-free, eukaryotic cell culture medium supplement according to claim 1, wherein said insulin substitute is selected from the group consisting of zinc chloride, zinc bromide, and zinc sulfate.7 water.

8. The serum-free, eukaryotic cell culture medium supplement according to claim 7, wherein said insulin substitute is zinc sulfate.7 water.

9. The serum-free, eukaryotic cell culture medium supplement formulation according to claim 1, wherein said amino acid ingredient comprises one or more amino acids selected from the group consisting of glycine, L-alanine, L-asparagine, L-cysteine, L-aspartic acid, L-glutamic acid, L-phenylalanine, L-histidine, L-isoleucine, L-lysine, L-leucine, L-glutamine, L-arginine, L-methionine, L-proline, L-hydroxyproline, L-serine, L-threonine, L-tryptophan, L-tyrosine, and L-valine, and derivatives thereof.

10. The serum-free, eukaryotic cell culture medium supplement according to claim 1, wherein said albumin substitute is selected from the group consisting of bovine pituitary extract, plant hydrolysate, fetal calf albumin (fetuin), egg albumin, human serum albumin (HSA), chick extract, bovine embryo extract, AlbuMAX® I, and AlbuMAX® II.

11. The serum-free, eukaryotic cell culture medium supplement according to claim 1, wherein said albumin substitute is AlbuMAX® I.

12. The serum-free, eukaryotic cell culture medium supplement according to claim 1, wherein said trace element ingredient comprises one or more trace element moieties selected from the group consisting of Ag⁺, Al³⁺, Ba²⁺, Cd²⁺, Co²⁺, Cr⁺, Ge⁴⁺, Se⁴⁺, Br⁻, I⁻, Mn²⁺, F⁻, Si⁴⁺, V⁵⁺, Mo⁶⁺, Ni²⁺, Rb⁺, Sn²⁺ and Zr⁴⁺.

13. The serum-free, eukaryotic cell culture medium supplement according to claim 1, wherein said supplement is concentrated.

14. The serum-free, eukaryotic cell culture medium supplement according to claim 1, wherein said supplement is concentrated from about 2-fold to about 10-fold.

15. The serum-free, eukaryotic cell culture medium supplement according to claim 1, wherein said supplement is added to a basal medium to a final concentration of about 0.5% to about 90%.

16. The serum-free, eukaryotic cell culture medium supplement according to claim 15, wherein said supplement is added to a basal medium to a final concentration of about 5% to about 50%.

17. The serum-free, eukaryotic cell culture medium supplement according to claim 16, wherein said supplement is added to a basal medium to a final concentration of about 5% to about 30%.

18. The serum-free, eukaryotic cell culture medium supplement according to claim 17, wherein said supplement is added to a basal medium to a final concentration of about 5% to about 20%.

19. The serum-free, eukaryotic cell culture medium supplement according to claim 18, wherein said supplement is added to a basal medium to a final concentration of about 15%.

20. A serum-free, eukaryotic cell culture medium supplement obtained by combining an albumin or an albumin substitute and one or more ingredients selected from group consisting of one or more amino acids, one or more vitamins, one or more transferrins or transferrin substitutes, one or more antioxidants, one or more insulins or insulin substitutes, one or more collagen precursors, and one or more trace elements,
wherein a basal cell culture medium supplemented with the supplement is capable of supporting the growth of embryonic stem cells in serum-free culture.

21. A serum-free, eukaryotic cell culture medium supplement comprising AlbuMAX® I, glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, sodium selenite, Ag⁺, Al³⁺, Ba²⁺, Cd²⁺, Co²⁺, Cr³⁺, Ge⁴⁺, Se⁴⁺, Br⁻, I⁻, Mn²⁺, F⁻, Si⁴⁺, V⁵⁺, Mo⁶⁺, Ni²⁺, Rb⁻, Sn²⁺ and Zr⁴⁺,
wherein a basal cell culture medium supplemented with said supplement is capable of supporting the growth of embryonic stem cells in serum-free culture.

22. A serum-free, eukaryotic cell culture medium supplement obtained by combining water, AlbuMAX® I, glycine, L-histidine.HClwater, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, sodium selenite, a Ag⁺ salt, an Al³⁺ salt, a Ba²⁺ salt, a Cd²⁺ salt, a Co²⁺ salt, a Cr³⁻ salt, a Ge⁴⁺ salt, a Se⁴⁺ salt, a Br⁻ salt, an I⁻ salt, a Mn²⁺ salt, a F⁻ salt, a Si⁴⁺ salt, a V⁵⁺ salt, a Mo⁶⁺ salt, a Ni²⁺ salt, a Rb⁺ salt, a Sn²⁺ salt, and a Zr⁴⁺ salt,
wherein each ingredient is present in an amount which, when added to a basal medium, supports the growth of embryonic stem cells in serum-free culture.

23. The serum-free, eukaryotic cell culture medium supplement according to claim 22, wherein said Ag⁺ salt is AgNO₃, said Al³⁺ salt is AlCl₃.6 water, said Ba²⁺ salt is Ba(C₂H₃O₂)₂, said Cd²⁺ salt is CdSO₄.8 water, said Co²⁺ salt is CoCl₂.6 water, said Cr³⁺ salt is Cr₂(SO₄)₃.1 water, said Ge⁴⁺ salt is GeO₂, said Se⁴⁺ salt is both Na₂SeO₃ and H₂SeO₃, said Br⁻ salt is KBr, said I⁻ salt is KI, said Mn²⁺ salt is MnCl₂.4 water, said F⁻ salt is NaF, said Si⁴⁻ salt is Na₂SiO₃.9 water, said V⁵⁺ salt is NaVO₃, said Mo⁶⁺ salt is (NH₄)₆Mo₇O₂₄.4 water, said Ni²⁺ salt is NiSO₄.6 water, said Rb⁺ salt is RbCl, said Sn²⁺ salt is SnCl₂, and said Zr⁴⁺ salt is ZrOCl₂.8 water.

24. A method of making a serum-free, eukaryotic cell culture medium supplement, said method comprising admixing water, AlbuMAX® I, glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, sodium selenite, a Ag⁺ salt, an Al³⁺ salt, a Ba²⁺ salt, a Cd²⁺ salt, a Co²⁺ salt, a Cr³⁺ salt, a Ge⁴⁺ salt, a Se⁴⁺ salt, a Br⁻ salt, an I⁻ salt, a Mn²⁺ salt, a F⁻ salt, a Si⁴⁺ salt, a V⁵⁺ salt, a Mo⁶⁺ salt, a Ni²⁺ salt, a Rb⁺ salt, a Sn²⁺ salt, and a Zr⁴⁺ salt,
wherein each ingredient is present in an amount which, when added to a basal medium, supports the growth of embryonic stem cells in serum-free culture.

25. The method according to claim 23, wherein said said Ag⁺ salt is AgNO₃, said Al³⁺ salt is AlCl₃.6 water, said Ba²⁺ salt is Ba(C₂H₃O₂)₂, said Cd²⁺ salt is CdSO₄.8 water, said Co²⁺ salt is CoCl₂.6 water, said Cr³⁺ salt is Cr₂(SO₄)₃.1 water, said Ge⁴⁺ salt is GeO₂, said Se⁴⁺ salt is both Na₂SeO₃ and H₂SeO₃, said Br⁻ salt is KBr, said I⁻ salt is KI, said Mn²⁺ salt is MnCl₂.4 water, said F⁻ salt is NaF, said Si⁴⁺ salt is Na₂SiO₃.9 water, said V⁵⁺ salt is NaVO₃, said Mo⁶⁺ salt is (NH₄)₆Mo₇O₂₄.4 water, said Ni²⁺ salt is NiSO₄.6 water, said Rb⁺ salt is RbCl, said Sn²⁺ salt is SnCl₂, and said Zr⁴⁺ salt is ZrOCl₂.8 water.

26. A serum-free eukaryotic cell culture medium comprising a basal cell culture medium supplemented with the serum-free cell culture supplement according to claim 1,
wherein said supplemented culture medium is capable of supporting the growth of embryonic stem cells in serum-free culture.

27. The serum-free eukaryotic cell culture medium according to claim 6, wherein said medium is a 1× medium formulation.

28. The serum-free eukaryotic cell culture medium according to claim 26, wherein said medium is a concentrated medium formulation.

29. The serum-free, eukaryotic cell culture medium according to claim 26, wherein the final concentration of said supplement is about 0.5% to about 90%.

30. The serum-free, eukaryotic cell culture medium according to claim 29 wherein the final concentration of said supplement is about 5% to about 50%.

31. The serum-free, eukaryotic cell culture medium according to claim 30, wherein the final concentration of said supplement is about 5% to about 30%.

32. The serum-free, eukaryotic cell culture medium according to claim 31, wherein the final concentration of said supplement is about 5% to about 20%.

33. The serum-free, eukaryotic cell culture medium according to claim 30, wherein the final concentration of said supplement is about 15%.

34. A serum-free eukaryotic cell culture medium obtained by combining a basal cell culture medium with the serum-free supplement according to claim 1,
wherein said medium is capable of supporting the growth of embryonic stem cells in serum-free culture.

35. A method of making a serum-free eukaryotic cell culture medium, said method comprising admixing a basal cell culture medium with the supplement according to claim 1,
wherein said medium is capable of supporting the growth of embryonic stem cells in serum-free culture.

36. The method according to claim 35, wherein said medium is a 1× formulation.

37. The method according to claim 35, wherein said medium is a concentrated formulation.

38. The serum-free, eukaryotic cell culture medium according to the method of claim 35, wherein the final concentration of said supplement is about 0.5% to about 90%.

39. The serum-free, eukaryotic cell culture medium according to the method of claim 38, wherein the final concentration of said supplement is about 5% to about 50%.

40. The serum-free, eukaryotic cell culture medium according to the method of claim 39, wherein the final concentration of said supplement is about 5% to about 30%.

41. The serum-free, eukaryotic cell culture medium according to the method of claim 40, wherein the final concentration of said supplement is about 5% to about 20%.

42. The serum-free, eukaryotic cell culture medium according to the method of claim 41, wherein the final concentration of said supplement is about 15%.

43. A composition comprising embryonic stem cells in a serum-free medium, wherein said serum-free medium is capable of supporting the growth of embryonic stem cells in serum-free culture.

44. The composition according to claim 43, wherein said medium is the medium according to claim 26 or 34.

45. The composition according to claim 44, wherein said composition is capable of being stored indefinitely at less than or equal to about −135° C.

46. The composition according to claim 45, wherein said embryonic stem cells are obtained from an animal selected from the group consisting of human, monkey, ape, mouse, rat, hamster, rabbit, guinea pig, cow, swine, dog, horse, cat, goat, sheep, bird, reptile, fish, and amphibian.

47. The composition according to claim 46, wherein said embryonic stem cells are obtained from an animal selected from the group consisting of mouse, cow, goat, and sheep.

48. The composition according to claim 47, wherein said embryonic stem cells are obtained from mouse.

49. A product of manufacture comprising a container means containing embryonic stem cells and the supplement according to claim 1.

50. A product of manufacture comprising a container means containing embryonic stem cells in the medium according to claim 26 or 34.

51. A product of manufacture comprising one or more container means, wherein a first container means contains the supplement according to claim 1, wherein optionally a second container means contains a basal medium, wherein optionally a third container means contains embryonic stem cells.

52. A product of manufacture comprising one or more container means, wherein a first container means contains the medium according to claim 26 or 34, wherein optionally a second container means contains embryonic stem cells.

53. The product of manufacture according to any one of claims 49-52, wherein said product of manufacture is in a frozen state.

54. A method of expanding embryonic stem cells in serum-free culture, said method comprising
(a) contacting said embryonic stem cells with the medium according to claim 26 or 34; and
(b) cultivating said embryonic stem cells under serum-free conditions suitable to facilitate the expansion said embryonic stem cells.

55. The method according to claim 54, wherein said method further comprises seeding said embryonic stem cells upon a layer of feeder cells.

56. A method of producing a transgenic animal, said method comprising
(a) cultivating embryonic stem cells in the medium according to claim 26 or 34;
(b) introducing a nucleic acid molecule into said embryonic stem cells;
(c) selecting a recombinant embryonic stem cell clone;
(d) expanding said recombinant embryonic stem cell clone to form a population;
(e) injecting an aliquot of said recombinant embryonic stem cell clonal population into a blastocyst;
(f) transferring said injected blastocyst into a host pseudopregnant female animal; and
(g) selecting transgenic offspring.

57. The method according to claim 56, wherein said cultivating further comprises
(a1) contacting said embryonic stem cells with the medium according to claim 26 or 34; and
(a2) cultivating said embryonic stem cells under serum-free conditions suitable to facilitate the expansion said embryonic stem cells in serum-free culture.

58. The method according to claim 57 wherein said method comprises seeding said embryonic stem cells upon a layer of feeder cells.

59. A method of producing a transgenic animal, said method comprising
(a) cultivating embryonic stem cells in the medium according to claim 26 or 34;
(b) introducing a nucleic acid molecule into said embryonic stem cells;
(c) selecting a recombinant embryonic stem cell clone;
(d) expanding said recombinant embryonic stem cell clone to form a population;
(e) co-culturing a small number of the embryonic stem cells with early stage embryos to form aggregates of embryos;
(f) transferring said aggregated embryos into a host pseudopregnant female animal; and
(g) selecting transgenic offspring.

60. The method according to claim 59, wherein said cultivating further comprises
(a1) contacting said embryonic stem cells with the medium according to claim 26 or 34; and
(a2) cultivating said embryonic stem cells under serum-free conditions suitable to facilitate the expansion said embryonic stem cells in serum-free culture.

61. The method according to claim 60 wherein said method comprises seeding said embryonic stem cells upon a layer of feeder cells.

62. A method of producing a recombinant protein from a transgenic animal, said method comprising
(a) cultivating embryonic stem cells in the medium according to claim 26 or 34;
(b) introducing a nucleic acid construct comprising a nucleic acid molecule which encodes a protein of interest encoding said protein into said embryonic stem cells;
(c) selecting a recombinant embryonic stem cell clone;
(d) expanding said recombinant embryonic stem cell clone to form a population of recombinant embryonic stem cells;
(e) injecting said recombinant embryonic stem cell clonal population into a blastocyst;
(f) transferring said injected blastocyst into a host pseudopregnant female animal;
(g) selecting transgenic offspring;
(h) raising said selected transgenic animal(s) under conditions suitable to promote the health of said transgenic animal; and
(i) isolating said recombinant protein from said transgenic animal.

63. The method according to claim 62, wherein said cultivating further comprises
(a1) contacting said embryonic stem cells with the medium according to claim 26 or 34; and
(a2) cultivating said embryonic stem cells under serum-free conditions suitable to facilitate the expansion of said embryonic stem cells in serum-free culture.

64. The method according to claim 63, wherein said method further comprises seeding said embryonic stem cells upon a layer of feeder cells.

65. A method of producing a recombinant protein from a transgenic animal, said method comprising
(a) cultivating embryonic stem cells in the medium according to claim 26 or 34;
(b) introducing a nucleic acid construct comprising a nucleic acid molecule which encodes a protein of interest encoding said protein into said embryonic stem cells;
(c) selecting a recombinant embryonic stem cell clone;
(d) expanding said recombinant embryonic stem cell clone to form a population of recombinant embryonic stem cells;
(e) co-culturing a small number of the embryonic stem cells with early stage embryos to form aggregates of embryos;
(f) transferring said aggregated embryos into a host pseudopregnant female animal; and
(g) selecting transgenic offspring;
(h) raising said selected transgenic animal(s) under conditions suitable to promote the health of said transgenic animal; and
(i) isolating said recombinant protein from said transgenic animal.

66. The method according to claim 67, wherein said method further comprises
(a1) contacting said embryonic stem cells with the medium according to claim 26 or 34; and
(a2) cultivating said embryonic stem cells under serum-free conditions suitable to facilitate the expansion of said embryonic stem cells in serum-free culture.

67. The method according to claim 66, wherein said method further comprises seeding said embryonic stem cells upon a layer of feeder cells.

68. A method for controlling or preventing the differentiation of embryonic stem cells in serum-free culture, said method comprising
(a) contacting said embryonic stem cells with the medium according to claim 26 or 34; and
(b) cultivating said embryonic stem cells under serum-free conditions suitable to control or prevent the differentiation of embryonic stem cells and facilitate the expansion of said embryonic stem cells in serum-free culture.

69. The method according to claim 68, wherein said method further comprises seeding said embryonic stem cells upon a layer of feeder cells.

70. The method according to claim 69, wherein said cultivating further comprises supplementing said medium with one or more factors which control or prevent the differentiation of said embryonic stem cells.

71. The method according to claim 70, wherein said factor is selected from the group consisting of leukemia inhibitory factor, steel factor, ciliary neurotrophic factor, and oncostatin M.

72. The method according to claim 71, wherein said factor is leukemia inhibitory factor.

73. The method according to claim 71, wherein said factor is steel factor.

74. The method according to claim 71, wherein said factor is ciliary neurotrophic factor.

75. The method according to claim 71, wherein said factor is oncostatin M.

76. A method of causing embryonic stem cells to differentiate into a particular type of cell in serum-free culture, said method comprising
(a) contacting said embryonic stem cells with the medium according to claim 26 or 34;
(b) cultivating said embryonic stem cells under conditions suitable to facilitate the expansion of embryonic stem cells in serum-free culture; and
(c) adding a differentiation factor or changing culturing conditions to induce differentiation of embryonic stem cells to form a different type of cell.

77. The method according to claim 76, wherein said method further comprises seeding said embryonic stem cells upon a layer of feeder cells.

78. The method according to claim 76, wherein said cultivating said embryonic stem cells under conditions suitable to prevent the differentiation of and facilitate the expansion of said cells further comprises supplementing said culture medium with one or more growth factors which prevent differentiation of said embryonic stem cells.

79. The method according to claim 76, wherein said cultivating said expanded embryonic stem cells further comprises supplementing said culture medium with one or more growth factors which facilitate differentiation of said embryonic stem cells.

80. A method of providing differentiated embryonic stem cells, in serum-free culture, to a mammal, said method comprising
(a) contacting embryonic stem cells with the medium according to claim 26 or 34;
(b) cultivating said embryonic stem cells under conditions suitable to facilitate the expansion of embryonic stem cells in serum-free culture;
(c) adding a differentiation factor or changing culturing conditions to induce differentiation of embryonic stem cells to form a different type of cell; and
(d) introducing said differentiated cells into a mammal.

81. The method according to claim 80, wherein said method further comprises seeding said embryonic stem cells upon a layer of feeder cells.

82. The method according to claim 80, wherein said cultivating said embryonic stem cells under serum-free conditions suitable to prevent the differentiation of said cells further comprises supplementing said culture medium with one or more factors.

83. The method according to claim 82, wherein said factor is leukemia inhibitory factor.

84. The method according to claim 80, wherein said cultivating said expanded embryonic stem cells under serum-free conditions suitable to induce the differentiation of said cells further comprises supplementing said culture medium with one or more growth factors.

85. A method of obtaining embryonic stem cells in serum-free culture, said method comprising
(a) isolating embryonic stem cells from blastocysts; and
(b) cultivating said isolated embryonic stem cells in the medium according to claim 26 or 34.

86. A method of producing recombinant protein embryonic stem cells in serum-free culture, said method comprising
(a) obtaining a recombinant embryonic stem cell containing a nucleic acid molecule which encodes a protein of interest;
(b) culturing said embryonic stem cell in serum free culture to form a population of recombinant embryonic stem cells; and
(c) isolating said protein from said embryonic stem cells or from the medium in which said cells are cultured.

87. The method according to claim 87, wherein said isolating further comprises
(c1) isolating said protein from said embryonic stem cells.

88. The method according to claim 86, wherein said isolating further comprises
(c1) isolating said protein from said harvested medium.
